(19) United States
(12) Patent Application Publication
(10) Pub. No.: US 2013/0072563 A1
(43) Pub. Date: Mar. 21, 2013

(54) PERACID AND 2-HYDROXY ORGANIC ACID COMPOSITIONS AND METHODS FOR SANITATION AND DISEASE PREVENTION

(71) Applicant: Kai Lai Grace Ho, Salinas, CA (US)

(72) Inventor: Kai Lai Grace Ho, Salinas, CA (US)

(73) Assignee: Fresh Express Incorporated, Salinas, CA (US)

(21) Appl. No.: 13/528,724

(22) Filed: Jun. 20, 2012

Related U.S. Application Data
(63) Continuation of application No. PCT/US2010/061361, filed on Dec. 20, 2010.
(60) Provisional application No. 61/288,753, filed on Dec. 21, 2009.

(51) Int. Cl. A61K 31/327; A61K 31/19

(52) U.S. Cl. CPC A61K 31/327; A61K 31/19
USPC 514/557

(57) ABSTRACT

Methods and compositions for treating living surfaces to control microorganisms are provided. The method treats produce by contacting a living surface with an aqueous solution comprising i) an organic peracid of the formula RC(O)OOH wherein R is methyl, ethyl, n-propyl, or s-propyl; ii) a 2-hydroxy organic acid selected from tartaric acid, citric acid, malic acid, mandelic acid, and lactic acid; and iii) water.

### BACKGROUND OF THE INVENTION

Safe and reliable means of removing microorganisms from the skin or living surfaces is a growing public health and agricultural concern. Existing methods for removing or reducing environmental microorganisms do not adequately control microorganisms that have the potential to cause disease or spoilage. Accordingly, there is a large need for new methods and compositions that can greatly reduce the presence of microorganisms in the environment.

This invention provides compositions and methods that meet these needs.

### BRIEF SUMMARY OF THE INVENTION

The invention relates to the discovery that an aqueous solution comprising peroxyacetic acid, lactic acid, and (optionally) sodium lauryl sulfate or another surfactant is surprisingly effective in reducing microbial contamination on surfaces. The combination of the ingredients is much more effective at reducing microbes than any one of the ingredients acting alone. Accordingly, the invention provides compositions and methods useful in the sanitation of surfaces. Sanitizing or disinfecting controls or reduces the presence of unwanted or harmful microorganisms. In a first aspect the invention provides methods of sanitizing or disinfecting skin and living surfaces by contacting the surface with a composition according to the invention.

The compositions according to the invention are aqueous solutions having a pH of 2.5 to 6.0 and comprising i) an organic peracid of the formula RC(O)OOH wherein R is methyl, ethyl, n-propyl, or s-propyl; ii) a 2-hydroxy organic acid selected from tartaric acid, citric acid, malic acid, mandelic acid, and lactic acid; and iii) water. Other ingredients (e.g., anionic surfactants) can also be used. In preferred embodiments, the peracid is peroxyacetic acid (also known as peracetic acid or acetyl hydroperoxide), the organic acid is lactic, acid (also known as 2-hydroxypropionic acid), and if present, the preferred anionic surfactant is sodium lauryl sulfate. Because aqueous sanitizing solutions of peracids may exist in equilibrium with, or be formed from concentrated solutions of, hydrogen peroxide, their corresponding acid, and water, the aqueous sanitizing solutions may also contain hydrogen peroxide and the corresponding acid (e.g., acetic acid in the case of peroxyacetic acid). The sanitizing solutions may be provided as concentrates or in ready-to-use aqueous formulations.

The compositions of the present invention can be incorporated with other ingredients to form a variety of sanitizing or disinfectant products including but not limited to hand cleansers, mouthwashes, surgical scrubs, body splashes, hand sanitizer gels and foams and sprays, disinfectant wipes, bath additives, and similar personal care products. Additional types of products include disinfectant foams, creams, mousses, and the like, and compositions containing organic and inorganic filler materials, such as emulsions, lotions, creams, pastes, and the like. The present antibacterial compositions can be manufactured as dilute ready-to- use compositions, or as concentrates that can be diluted prior to use. The various products in which the disinfectants are used may also include colorants, fragrances, and/or flavorants, depending on the nature of the product. Further, gels or aerosols may also be fragranced for similar or other reasons.

Because these compositions can be formulated to be very safe, e.g., often including low-residual, non-toxic, food grade components, for humans, animals and plants. These compositions can be topically applied to humans or animals or the surface of living plants. For example, these compositions, in human applications, can be used for skin and wound antiseptics, burn treatments, diaper rash products, and other skin care products. Additionally, these compositions can be used inside the mouth, such as for mouthwashes, toothpastes, and various other disinfecting solutions that are be employed in dental mold materials. As dental molds are known to spread significant disease in the dental industry, such use with dental molds can prevent or reduce the spread of pathogens from a patient's mouth to lab employees working with the finished molds. Still a further category of use includes application for topical antibiotic and antiviral purposes. In still other embodiments, the compositions provide the bulk fluid for a foot bath, nalon salon solutions, a soak tub, a spa, ultrasound treatment tub, wading pools, swimming pools. In these embodiments, the compositions can serve to sanitize both the skin or muscosa of a subject in contact with a composition according to the invention.

In another aspect, the invention provides the compositions according to the invention in a packaging or format suitable for use in a method according to the invention.

### BRIEF DESCRIPTION OF THE DRAWINGS

FIG. 1 is a comparison of five treatments, in left to right order: a) chlorinated water: 50-70 ppm active chlorine at pH 6.5; b) CS: a commercial antimicrobial produce cleaner with major active ingredients as citric acid plus surfactants; c) Peroxyacetic acid: 70 to 80 ppm peroxyacetic acid +0.01% surfactant; d) lactic acid solution: 0.9 to 1.2% lactic acid +0.01% surfactant; and e) FE: 70 to 80 ppm peroxyacetic acid +0.9 to 1.2% lactic acid +0.01% surfactant) on flume-water suspended cells challenge test. The surfactant used was sodium lauryl sulfate.

FIG. 2 is a comparison of each of the five treatments of FIG. 1 in a leaf-attached cell challenge test.

FIG. 3 is a comparison of the ability of chlorinated water and an aqueous solution according to the invention (FE: peroxyacetic acid, lactic acid and sodium lauryl sulfate) to reduce the decay of treated produce.

FIG. 4 is a comparison of the ability of chlorinated water and an aqueous solution according to the invention (FE: peroxyacetic acid, lactic acid and sodium lauryl sulfate) to reduce off-odor in treated produce.

FIG. 5 is a comparison of the ability of chlorinated water and an aqueous solution according to the invention (peroxyacetic acid, lactic acid and sodium lauryl sulfate) to reduce the decay of Spring Mix with a low-moisture content.

FIG. 6 is a comparison of the ability of treatment with chlorinated water or an aqueous solution according to the invention (peroxyacetic acid, lactic acid and sodium lauryl sulfate) to reduce off-odor in a Spring Mix with a low-moisture content.

FIG. 7 is a comparison of the ability of chlorinated water and an aqueous solution according to the invention (peroxyacetic acid, lactic acid and sodium lauryl sulfate) to inhibit the growth of indigenous microorganisms in a Spring Mix with a low-moisture content.

FIG. 8 is a comparison of the ability of chlorinated water and an aqueous solution according to the invention (peroxyacetic acid, lactic acid and sodium lauryl sulfate) to inhibit spoilage in a Spring Mix with a low-moisture content.

FIG. 9 is a comparison of the ability of chlorinated water and an aqueous solution according to the invention (peroxyacetic acid, lactic acid and sodium lauryl sulfate) to reduce the decay of Spring Mix with a high-moisture content.

FIG. 10 is a comparison of the ability of treatment with chlorinated water or an aqueous solution according to the invention (peroxyacetic acid, lactic acid and sodium lauryl sulfate) to reduce off-odor in a Spring Mix with a high-moisture content.

FIG. 11 is a comparison of the ability of chlorinated water and an aqueous solution according to the invention (peroxyacetic acid, lactic acid and sodium lauryl sulfate) to inhibit growth of indigenous microorganisms in a Spring Mix with a high-moisture content.

FIG. 12 is a comparison of the ability of chlorinated water and an aqueous solution according to the invention (peroxyacetic acid, lactic acid and sodium lauryl sulfate) to inhibit spoilage in a Spring Mix with a high-moisture content.

FIG. 13 is a comparison of the ability of chlorinated water and an aqueous solution according to the invention (peroxyacetic acid, lactic acid and sodium lauryl sulfate) to reduce the decay of spinach.

FIG. 14 is a comparison of the ability of treatment with chlorinated water or an aqueous solution according to the invention (peroxyacetic acid, lactic acid and sodium lauryl sulfate) to reduce off-odor in spinach.

FIG. 15 is a comparison of the ability of chlorinated water and an aqueous solution according to the invention (peroxyacetic acid, lactic acid and sodium lauryl sulfate) to inhibit the growth of indigenous microorganisms in spinach with a high-moisture content.

FIG. 16 is a comparison of the ability of chlorinated water and an aqueous solution according to the invention (peroxyacetic acid, lactic acid and sodium lauryl sulfate) to inhibit spoilage microorganisms in spinach.

### DETAILED DESCRIPTION OF THE INVENTION

The invention relates to the discovery that an aqueous composition comprising peroxyacetic acid, lactic acid, and (optionally) sodium lauryl sulfate is surprisingly effective in reducing microbial contamination on surfaces. The combination of the ingredients is much more effective at reducing attached microbes on an item than any one of the ingredients acting alone.

Peroxyacetic acid antimicrobial activity relies on its high oxidizing potential. The mechanism of oxidation is the transfer of electrons, therefore the stronger the oxidizer, the faster the electrons are being transferred to the microorganism and the faster the microorganism is inactivated or killed. Therefore based on the table below peroxyacetic acid has a higher oxidation potential than chlorine sanitizers but less than that of ozone.

Oxidation Capacity of Selected Sanitizers

| Sanitizer | eV* |
|---|---|
| Ozone | 2.07 |
| Peroxyacetic acid | 1.81 |
| Chlorine Dioxide | 1.57 |
| Sodium hypochlorite (Chlorine bleach) | 1.36 |

*electron-Volts

As diffusion of the molecule is slower than its half-life, peroxyacetic will react with any oxidizable compounds in its vicinity. It can damage virtually all types of macromolecules associated with a microorganism; for e.g. carbohydrates, nucleic acids (mutations), lipids (lipid peroxidation) and amino acids (e.g. conversion of Phe to m-Tyr and o-Tyr), and ultimately lysis the cell. Conventionally 2-hydroxy organic acids such as lactic acid that possess the chemical properties of oxidizable organic compounds would be taught away from being used together with a strong oxidizer, particularly with reference to peracids. Hence, it is particularly surprising to combine the peracetic acid and lactic acid in this invention and shown that the two compounds have synergistic effects rather than one counteracting against the other.

Definitions

It must be noted that, as used in this specification and the appended claims, the singular forms “a,” “an” and “the” include plural referents unless the content clearly dictates otherwise. Thus, for example, reference to a “surfactant” includes two or more such surfactants.

Unless defined otherwise, all technical and scientific terms used herein have the same meaning as commonly understood by one of ordinary skill in the art to which the invention pertains. All ranges are inclusive of the end values.

With reference to the aqueous solutions and methods of the invention, “peracid” and “organic peracid” refer to compounds of the structure RC(O)OOH in which R is an aliphatic group having from 1 to 3 carbon atoms. R may be methyl, ethyl, n-propyl, or s-propyl. A particularly preferred peracid is peracetic acid/peroxyacetic acid/PAA/(CH₃C(O)OOH). Mixtures of the above organic peracids may be used.

In aqueous solutions, organic peracids exist in a chemical equilibrium with hydrogen peroxide and accordingly can be formed from the corresponding organic acids and hydrogen peroxide in the reaction:

RCOOH+H₂O₂ [symbol] RC(O)OOH+H₂O

The equilibrium concentration of each reactant can be calculated from the equilibrium equation:

([RCOOOH][H₂O])/([RCOOH][H₂O₂])=Kₐₚ (Eq. 1)

wherein: [RCOOOH] is the concentration of peracid in mole/L; [H₂O] is the concentration of water in mole/L; [RCOOH] is the concentration of organic acid in mole/L; and [H₂O₂ ] is the concentration of hydrogen peroxide in mole/L; and Kₐₚ is the apparent equilibrium constant for the peracid equilibrium reaction (Equation I).

The apparent equilibrium constant, Kₐₚ, varies with both the peracid chosen and with temperature. Equilibrium constants for peracid formation can be found in D. Swern, ed., Organic Peroxides, Vol. 1, Wiley-Interscience, New York, 1970. At a temperature of 40° C., the apparent equilibrium constant for peroxyacetic acid is about 2.21. In accordance with this equilibrium reaction, organic peracid solutions comprise hydrogen peroxide and the corresponding organic acid in addition to the organic peracid.

When diluted, a relatively long period of time may lapse before a new equilibrium is achieved. For instance, equilibrium solutions that comprise about 5% peroxyacetic acid typically comprise about 22% hydrogen peroxide. Equilibrium solutions that comprise about 15% peroxyacetic acid typically comprise about 10% hydrogen peroxide. When these equilibrium solutions are diluted to solutions that comprise about 50 ppm of peroxyacetic acid, the solution produced by dilution of the 5% peroxyacetic acid solution comprises about 220 ppm of hydrogen peroxide, and the solution produced by dilution of 15% solution comprises about 33 ppm of hydrogen peroxide. Accordingly, in some embodiments, the sanitizing solution is provided as a concentrate which is diluted to the desired peracid concentration with water or with an aqueous solution comprising other components of the sanitizing solution according to the invention just prior to use. In some embodiments, the sanitizing solutions are provided as concentrates which are diluted just prior to use.

Peracids are readily commercially available in accordance with the above equilibrium. Peroxyacetic acid (CAS No. 79-21-0) is readily commercially available, for instance, as aqueous solution comprising peroxyacetic acid (35%), hydrogen peroxide (6.5%), acetic acid 64-19-7 (40%), sulfuric acid (about 1%) and water (about 17%) (all units w/w).

The 2-hydroxy organic acid is selected from tartaric acid, citric acid, malic acid, mandelic acid, and lactic acid. The predominant biological optical isomers are preferred. The 2-hydroxy organic acid can also be provided as the racemate, as well as any of its optically pure isomers. In some embodiments, the (+) enantiomer is preferred (e.g., L-lactic acid, L(+)-Lactic acid).

As used herein, the term “sanitize” or “disinfect” shall mean the reduction of viable microorganisms on surfaces with the exception of bacterial endospores. In some embodiments, the reduction is by at least 90%, 99%, 99.9%, 99.99%, 99.999% (e.g., by 1, 2 3, 4, or 5 log units, respectively) or at least by 2, 3, 4, 5, 6, 7, 8, or log units as measured before and after contact with the sanitizing solutions according to the invention.

As used herein, the term “living surface” external surfaces of a human, animal, or plant and includes the skin, hair, feathers, nails, orifices of humans and animals, and the leaves stems, seeds, flowers, and root balls of living plants. Living surfaces also includes the mucosal and other inner surfaces of the oral cavity of animals and humans. Skin includes, but is not limited to, the skin of the hands, feet, face, scalp, torso, and limbs.

The term “essentially free” means that the referenced compound or substance is present in the solution at a level less than about 300, preferably less than about 150 and more preferably less than about 50 and most preferably less than about 10 ppm or even 1 ppm by weight.

Compositions of the Invention

Accordingly, in a first aspect, the invention provides a composition (e.g. aqueous solution or gel) comprising 1) an organic peracid of the formula RC(O)OOH wherein R is methyl, ethyl, n-propyl, or s-propyl; ii) a 2-hydroxy organic acid selected from tartaric acid, citric acid, malic acid, mandelic acid, and lactic acid; and iii) water. Optionally, iv) an anionic surfactant may be included. In some embodiments, the aqueous solution may have a neutral pH of from 6.8 to 7.8 or 7.2 to 6.6, or about 7.4). In other embodiments, the pH is from 2.5 to 6.0, 2.5 to 3.5, 2.5 to 4.0, 2.7 to 3.5, 2.5 to 5.0, 3.0 to 4.0, 3.0 to 5.0, 3.0 to 6.0, or from 3.5 to 4.5.

Suitable 2-hydroxy organic acids for use in the aqueous solutions of the invention are tartaric acid, citric acid, malic acid, mandelic acid, and lactic acid (i.e., 2-hydroxypropanoic acid). An exemplary 2-hydroxy organic acid is lactic acid. A combination of two or more of any of the above 2-hydroxy organic acids may be used (e.g., lactic acid +citric acid; lactic acid +tartaric acid; lactic acid +malic acid; lactic acid +mandelic acid;).

A sanitizing composition according to the invention comprises i) an organic peracid of the formula RC(O)OOH wherein R is methyl, ethyl, n-propyl, or s-propyl; ii) a 2-hydroxy organic acid selected from tartaric acid, citric acid, malic acid, mandelic acid, and lactic acid; and a pH from 2.5 to 7.8, inclusive, wherein the concentration of peracid is from 40 to 250 ppm (w/w) inclusive, and the concentration of the 2-hydroxy organic acid is from 0.1 to 1% (w/w), inclusive. In some further embodiments of any of the above, the principal component by weight of the composition is water. In some embodiments, the composition according to the invention is at least 50%, 60%, 70%, 80%, 90%, 95%, 98% or 99% water by weight.

In some embodiments, the peracid is peroxyacetic acid, the organic acid is lactic acid, and the anionic surfactant is sodium lauryl sulfate. In other embodiments, the concentration of peracid acid in the solution is from 3 to 100 ppm (w/w), the concentration of 2-hydroxy organic acid in the solution is from 0.1% to 2% (w/w); and the pH is between 2.5 and 5.0. In a still further embodiment, the concentration of peracid is 5 to 100 ppm (w/w), the concentration of 2-hydroxy organic acid is 0.1 to 2% (w/w). In alternative embodiments of the above, the aqueous solution may have a neutral pH of from 6.8 to 7.8 or 7.2 to 6.6, or about 7.4).

In an additional embodiment, the aqueous solution of the invention, has a concentration of peracid in the solution from about 60 to 80 ppm (w/w), a concentration of 2-hydroxy organic acid in the solution of from about 0.2% to 1.25% (w/w); and a pH between about 2.8 to 4.2 or 3.8 and 4.2, inclusive. In alternative embodiments of the above, the aqueous solution may have a neutral pH of from 6.8 to 7.8 or 7.2 to 6.6, or about 7.4).

In some embodiments, the concentration of the peracid in the solution can be from 3 to 100 ppm (w/w), the concentration of 2-hydroxy organic acid in the solution from 0.1% to 2% (w/w); and the pH is between 2.5 and 5.0. In a still further embodiment, the concentration of peracid is 50 to 100 ppm (w/w) and the concentration of 2-hydroxy organic acid is 0.1 to 1% (w/w). In further embodiments, the peracid is peroxyacetic acid and the 2-hydroxy organic acid is lactic acid (e.g., L(+)-lactic acid). In still further embodiments, the concentration of the peracetic acid is 60 to 90 ppm or 70 to 80 ppm. In still further embodiments of such, the concentration of the lactic acid is 0.1 to 0.8% or 0.2 to 0.4%(w/w). In alternative embodiments of the above, the aqueous solution may have a neutral pH of from 6.8 to 7.8 or 7.2 to 6.6, or about 7.4).

In a particularly preferred embodiment, the invention provides a composition comprising, or consisting essentially of, an aqueous solution of peroxyacetic acid and lactic acid (e.g., L-(+)-Lactic acid) at a pH of from about 2.5 to 6.0, and more preferably at a pH between 2.8 to 4.2 or 3.8 to 4.2, inclusive, wherein an amount of the solution further comprises hydrogen peroxide and acetic acid and the composition is substantially free of any surfactant. In some embodiments, the aqueous solution is substantially free of any isomer of lactic acid other than L-(+)-Lactic acid. In further embodiments of any of the above, the concentration of peracid (e.g., peroxyacetic acid) in the solution is from 30 to 300 ppm (w/w), 60 to 80 ppm (w/w), 50 to 200 ppm (w/w); 60 to 160 ppm (w/w), 120 to 160 ppm (w/w), or 140 to 160 ppm (w/w); and the concentration of 2-hydroxy-organic acid (e.g., lactic acid) in the solution is selected from 0.1% to 5% (w/w), 0.1% to 2%, 0.2% to 1%, 0.2% to 0.6%, or 0.1% to 0.5%, or about 2%, 3%, or 4%; and the pH is from between 2.5 and 6.0, 2.5 to 5.0, 2.8 and 3.2, 2.5 and 3.5, or 2.6 and 3.2. In other embodiments of the above the solution is for contacting the surface to be sanitized from 10, 20 or 30 seconds to 2 minutes or about 10, 20, 30 or 40 secs. In further embodiments, the concentration of peracid acid is from 30 to 100 ppm (w/w), and the concentration of the 2-hydroxy organic acid is from 0.3 to 2.0% (w/w). In a particularly preferred embodiment, the concentration of peracid is 70 to 80 ppm (w/w), and the concentration of the 2-hydroxy organic acid is from 0.2 to 0.4% (w/w). In other embodiments of any of the above, the solution is at a temperature of 35° F. to 45° F. or at ambient temperature. These aqueous solutions can be free or substantially free of surfactants including any or all of nonionic surfactants, cationic surfactants or anionic surfactants. Generally, low levels of hydrogen peroxide from 1 to 20 ppm, 5 to 15 ppm, or 7 to 12 ppm may be present in the solution. In some embodiments, any peracid of the 2-hydroxy organic acid formed from hydrogen peroxide or present in the aqueous solution can be present in an amount which is less than 1/10^{th}, ⅕^{th} , 1/20^{th} , or 1/50^{th} the amount of the corresponding 2-hydroxyorganic acid in the solution. In preferred embodiment of the above, the peracid is peroxyacetic acid and the 2-hydroxyorganic acid is selected from one or more of tartaric acid, citric acid, malic acid, mandelic acid, and lactic acid. In a particularly preferred embodiment, of any of the above, the 2-hydroxy organic acid is lactic acid.

In alternative embodiments of any of the above, the aqueous solution may have a neutral pH of from 6.8 to 7.8 or 7.2 to 6.6, or about 7.4.

In some embodiments, the composition comprises an amine oxide at a mole ratio of amine oxide to peroxycarboxylic acid of 1 or more. Many peroxycarboxylic acid composition exhibit a sharp, annoying, or otherwise unacceptable odor. Such an unacceptable odor can be reduced by adding an amine oxide to the peroxycarboxylic acid. The peroxycarboxylic acid can be made in the presence of the amine oxide, or the amine oxide can be added after forming the peroxycarboxylic acid. In an embodiment, the amine oxide can be employed in food products or for cleaning or sanitizing food processing equipment or materials. In an embodiment, the amine oxide can be employed in a health-care environment. In an embodiment, the amine oxide is non-toxic. In an embodiment, the amine oxide can be employed according to guidelines from government agencies, such as the Food and Drug Administration, without adverse labeling requirements, such as labeling with a skull and cross bones or the like. Preferred amine oxides include octyl amine oxide (e.g., octyldimethylamine oxide), lauryldimethyl amine oxide, and the like. Alternatively, the amine oxide can be applied separately to a surface previously treated with a composition of the invention. In such embodiments, the amine oxide is preferably in an aqueous solution.

The amine oxide is typically present in a quantity that effectively reduces odor of the peroxycarboxylic acid. Suitable levels of amine oxide include a mole ratio of amine oxide to peroxycarboxylic acid of 1 or more. In an embodiment, the mole ratio is greater than or equal to 2. In an embodiment, the mole ratio is greater than or equal to 3. In an embodiment, the mole ratio is 2 to 5. In an embodiment, the mole ratio is 3 to 5. Octyl dimethyl amine oxide has a molecular weight of about 3 (e.g. 2.7) times as great as peroxyacetic acid, and applicable weight ratios can be calculated on such a basis (see, U.S. Pat. No. 7,622,606, issued Nov. 24, 2009, which is incorporated by reference with respect to suitable amine oxides for this purpose).

Exemplary amine oxides are of the formula

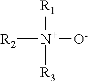

wherein R₁, R₂, and R₃ are independently selected from saturated or unsaturated and straight or branched alkyl groups having from 1-18 carbons and aromatic groups, etc. and which can optionally contain O, N or P as a heteroatom or polyalkoxy groups. Examples of amine oxides include, but are not limited to: alkyldimethylamine oxide, dialkylmethylamine oxide, alkyldialkoxyamine oxide, dialkylalkoxyamine oxide, dialkyletheramine oxide and dialkoxyetheramine oxide. In an embodiment, R₁ is an alkyl group having 4-18 carbons and R₂ and R₃ are alkyl groups having 1-18 carbons. In an embodiment, R₁ is an alkyl group having 6-10 carbons and R₂ and R₃ are alkyl groups having 1-2 carbons. In an embodiment, R₁ is an alkyl group having 8 carbons (an octyl group) and R₂ and R₃ are alkyl groups having 1-2 carbons. In an embodiment, R₁ is an alkyl group having 12 carbons (a lauryl group) and R₂ and R₃ are alkyl groups having 1-2 carbons. In some embodiments, the amine oxide is octyldimethylamine oxide, myristyldimethylamine oxide, didecylmethylamine oxide, methylmorpholine oxide, tetradecyldiethoxyamine oxide, or lauryldimethylamine oxide.

In some embodiments, accordingly, the peracid is peroxyacetic acid, the organic acid is lactic acid, and the anionic surfactant is sodium lauryl sulfate. In other embodiments, the concentration of peracid acid in the solution is from 3 to 100 ppm (w/w), the concentration of 2-hydroxy organic acid in the solution is from 0.1% to 2% (w/w); and the concentration of the anionic surfactant in the solution is from 10 to 2500 ppm, and the pH is between 2.5 and 5.0 or neutral(e.g., 6.8 to 7.8 or 7.2 to 6.6, or about 7.4).. In a still further embodiment, the concentration of peracid is 5 to 100 ppm (w/w), the concentration of 2-hydroxy organic acid is 0.1 to 2% (w/w), and the concentration of anionic surfactant is 50 to 400 ppm.

Generally, the concentration of hydrogen peroxide in the aqueous solutions is 5-fold to 10-fold less that the concentration of the peracid and its presence may reflect the equibilibrium or interconversion of the peracid with the corresponding acid and hydrogen peroxide. The concentration of the hydrogen peroxide can be for instance less than 5 ppm, 10 ppm or 20 ppm depending upon the selection and concentration of the peracid. Accordingly, the concentration of hydrogen peroxide in the aqueous solution is typically much less than that of the peracid.

Accordingly, in some embodiments, the invention provides an aqueous solution comprising i) an organic peracid of the formula RC(O)OOH wherein R is methyl, ethyl, n-propyl, or s-propyl; ii) a 2-hydroxy organic acid selected from tartaric acid, citric acid, malic acid, mandelic acid, and lactic acid; and, optionally, iii) an anionic surfactant; wherein the aqueous solution has a pH from 2.5 to 6.0, 4.0 to 6.0, 3.5 to 4.5, 3.0 to 5.0, 3.6 to 4.2, from 2.5 to 5.0, 2.5 to 4.5, 2.5 to 3.5, 2.7 to 3.5, 3.6 to 4.6, 2.8 to 3.2, inclusive, or about 3.0 (e.g., 3.0 +/−0.2; 3.0 +/−0.3); and the concentration of peracid is from 40 to 250 ppm (w/w) inclusive, and the concentration of the 2-hydroxy organic acid is from 0.1 to 1% (w/w), inclusive. In further embodiments, the aqueous solution has a peracid which is peroxyacetic acid and a 2-hydroxy organic acid which is is L-(+)-lactic acid. In still further embodiments, the concentration of the peroxyacetic acid in the solution is from 50 to 100 ppm (w/w), the concentration of the lactic acid in the solution is from 0.1% to 0.6% (w/w). A preferred aqueous solution has a concentration of peroxyacetic acid from 60 to 80 ppm (w/w) and a concentration of lactic acid of from 0.1% to 0.4% (w/w). In other embodiments of any of the above the composition has a neutral pH (e.g., of from 6.8 to 7.8 or 7.2 to 6.6, or about 7.4) or falls in a range selected from 2.5 to 4.5, 2.8 to 3.2, 2.5 to 5.0, and 2.7 to 3.5. In other embodiments of any of the above, the solution is at a temperature of 35° F. to 45° F. or at body or ambient temperature, or therebetween. These aqueous solutions can be substantially free of surfactants including any or all of nonionic surfactants, cationic surfactants or anionic surfactants. Generally, low levels of hydrogen peroxide from 1 to 20 ppm, 5 to 15 ppm, or 7 to 12 ppm may be present in the solution. Any peroxy 2-hydroxy organic acid formed or present in the aqueous solution can be present in an amount which is less than 1/10th, ⅕th 1/20^{th} , or 1/50^{th} the amount of the corresponding 2-hydroxyorganic acid in the solution. In alternative embodiments of the above, the aqueous solution may have a neutral pH of from 6.8 to 7.8 or 7.2 to 6.6, or about 7.4).

In some embodiments, the aqueous solution is formed by adding a solution of the 2-hydroxy organic acid which is substantially free of hydrogen peroxide to a solution of the peracid or by adding a solution of the peracid to a solution of the 2-hydroxy organic acid which is substantially free of hydrogen peroxide. The resulting mixture can be a concentrate or pre-blend as described above or in a sanitizing concentration suitable for contacting with a living surface to be treated as described herein. In other embodiments, the organic acid which is substantially free of any hydrogen peroxide and the peracid are added separately to an aqueous fluid used to wash or sanitize the living surface. In some embodiments, the pH and/or the concentration of the peracid and/or the concentration of the 2-hydroxy organic acid in the solution is maintained by monitoring one or more of the pH, concentration of the peracid, concentration of the 2-hydroxy organic acid, or oxidation reduction potential of the solution and adding a concentrate or pre-blend of the aqueous solution to maintain the pH, the concentration of the peracid and lactic acid in the aqueous solution during use of the solution in contacting the surface to be treated.

Any of the above solutions of the invention may in particular further comprise an agent to reduce or suppress sudsing or foaming of the solution during use or contact with the surface. The solutions according to the invention may also be essentially free of any nonionic and/or cationic surfactant and/or also be essentially free of any thickening agent.

In an additional embodiment, the aqueous solution of the invention has a concentration of peracid in the solution from about 60 to 80 ppm (w/w), a concentration of 2-hydroxy organic acid in the solution of from about 0.2% to 1.25% (w/w); and a concentration of anionic surfactant in the solution of from about 150 to 200 ppm (w/w), and a pH between about 3.8 and 4.2, inclusive or 3.8 and 4.2, inclusive.

In an additional embodiment, the aqueous solution of the invention has a concentration of peracid in the solution from about 60 to 80 ppm (w/w), a concentration of 2-hydroxy organic acid in the solution of from about 0.2% to 1.25% (w/w); a neutral pH of from 6.8 to 7.8 or 7.2 to 6.6, or about 7.4.

The aqueous solutions according to the invention may also optionally include a sequestering agent that chelates metals that catalyze the decomposition of hydrogen peroxide. These agents include, but are not limited to, organic phosphonic acids capable of sequestering bivalent metal cations, as well as the water-soluble salts of such acids. A common chelant is 1-hydroxyethylidene-1,1-diphosphonic acid. The chelants present in the sanitizer composition are typically diluted upon use, thus minimizing their effect during use. In particular, an aqueous sanitizer solution of the invention can optionally contain an agent to chelate magnesium or calcium.

Without being wed to theory, the presence of the optional anionic surfactant may serve to reduce the surface tension and viscosity of the aqueous solution and facilitate the spread of the solution over the surface being treated. The low viscosity improves the completeness of the treatment by promoting spreading over the surface of the food, especially where there are layers, rugosities, etc. The low viscosity also improves rinsing properties and the speed of any residual drying.

In some embodiments, the aqueous solution is capable of reducing a microbial contamination on the treated surface by at least 1 or 2 log units, more preferably, by at least 3 log units, and still more preferably by at least 4, log units according to any method as described in the Examples (e.g., using E. Coli or Listeria pathogen surrogates attached to lettuce leaves).

The solutions may be provided as a pre-blend or concentrate which is diluted with water to achieve a sanitizing solution as described herein. Pre-blends or concentrates are contemplated which require a 4- to 200-fold, 10 to 100-fold, 10 to 50-fold, 10 to 25 fold, 4 to 10-fold dilution with water before use (e.g., about a 5-, 10-, 20- 40-, 50, 100-fold dilution).

The term “substantially free” generally means the referenced substance is absent or present as a minor constituent which may not materially change the properties of the referenced material. With respect to hydrogen peroxide, a 2-hydroxy organic acid solution which is substantially free of hydrogen peroxide can be one which has no hydrogen peroxide or else has an amount of hydrogen peroxide which is less than 0.1 ppm (w/w). With respect to a peroxy 2-hydroxyorganic acid, a sanitizing solution is substantially free of the 2-hydroxy organic peracid if the 2-hydroxy organic peracid is absent in a referenced composition or is present in an amount which is less than 1/10^{th}, 1/20^{th}, 1/40^{th} or 1/100^{th} of that of the corresponding 2-hydroxy organic acid or is present only as a reaction product first formed by a reaction of the 2-hydroxy organic acid in solution containing hydrogen peroxide and an organic peracid of the formula RC(O)OOH wherein R is methyl, ethyl, n-propyl, or s-propyl. Accordingly, in some embodiments, the sanitizing composition or 2-hydroxy organic acid solution used in the making of the sanitizing composition is substantially free of a peracid of the 2-hydroxy organic acid.

The disinfectant or sanitizing compositions of the present invention can be incorporated with other ingredients to form a variety of products including sanitizer gels and foams, and disinfectant wipes. Additional types of products include disinfectant foams, creams, mousses, and the like, and compositions containing organic and inorganic filler materials, such as emulsions, lotions, creams, pastes, and the like. The present compositions can be manufactured as dilute ready-to-use compositions, or as concentrates that can be diluted prior to use. The various products in which the disinfectants are used may also include fragrances, depending on the nature of the product.

In one embodiment of the present invention, the disinfectant compositions are used to make disinfectant wipes. The disinfectant wipes of the present invention can be used to clean a variety of living surfaces. The wipes of the present invention can be made of a variety of fabrics. For the purposes of the present invention, fabrics can include cloths and papers, as well as woven and non-woven materials. The woven or nonwoven fabrics can be made of suitable materials such as rayon, nylon, or cotton, linen, combinations thereof. Examples of nonwoven fabrics are described in U.S. Pat. Nos. 3,786,615; 4,395,454; and 4,199,322; which are hereby incorporated by reference. The fabrics or papers can be impregnated with the disinfectant solution by any method known in the art. The wipes can be packaged individually or in any manner known in the art including individual blister-packs or wrapped or stacked multi-packs.

In another embodiment, the disinfectant composition of the present invention is formulated into a gel or gelatinous sanitization composition. In addition to the disinfectant compositions, the gel sanitizers of the present invention can include a thickening or gelling agent, wherein “thickening agent” and “gelling agent” are used interchangeably. For the purposes of the present invention, the terms “gel” or “gelatinous” sanitization compositions refers to a disinfectant liquid substances that can have a viscosity from about 1,000 centipoise to about 100,000 centipoise, or from 2,000 centipoise to 50,000 centipoise in another embodiment, though these ranges are not intended to be limiting. A hand gel may be considerably less viscous than a gel used for industrial cleaning or disinfectant purposes. Examples of gelling or thickening agents include but are not limited to natural gum such as guar and guar derivatives, a synthetic polymer, an acrylate homopolymer, an acrylate copolymer, a carbomer, cellulose, a cellulose derivative, algin, an algin derivative, a water-insoluble C₈-C₂₀ alcohol, carrageenan, a clay, an oil, a wax, aloe vera gel, fumed silica, mixtures thereof, and the like. The gelling agent can be present in the gelatinous sanitation composition in an amount from about 0.1 wt % to 50 wt % of the gelatinous composition. In another embodiment, the gelling agent is present in an amount from 0.25 wt % to 10 wt % of the gelatinous composition. The amount of gelling agent can be dependent on a variety of factors including the type of gelling agent and the desired viscosity of the gel. The gelatinous sanitizers can be used for a variety of applications. In one particular embodiment, the disinfectant composition can be mixed with natural aloe gel to form a disinfectant aloe formulation. Such formulations are especially favored where skin contact may occur or is intented.

In another embodiment, the disinfectant composition of the present invention can be formulated into a disinfectant foam or foaming composition. The disinfectant foams or foaming compositions include the disinfectant composition and foaming agents. Any foaming agent known in the art can be used depending on the desired application and characteristics of the resulting disinfectant foam.

Compositions may be formulated in various ways known in the art for administration purposes. To prepare the compositions of the present invention, an effective amount of the particular compound, in base or acid salt form, as the active ingredient is combined with one or more pharmaceutically-acceptable carriers and delivery vehicles. Numerous pharmaceutically acceptable carriers and delivery vehicles exist that are readily accessible and well-known in the art, which may be employed to generate the preparation desired (i.e. that permit administration of the composition topically. Representative examples of pharmaceutically acceptable carriers and delivery vehicles include aluminum stearate, lecithin, buffer substances such as the various phosphates, glycine, sorbic acid, potassium sorbate, partial glyceride mixtures of saturated vegetable fatty acids; water, salts or electrolytes, such as protamine sulfate, disodium hydrogen phosphate, potassium hydrogen phosphate, sodium chloride, and zinc salts; colloidal silica, magnesium trisilicate, polyvinyl pyrrolidone, cellulose-based substances, polyethylene glycol, sodium carboxymethylcellulose, polyarylates, waxes, polyethylene, polyoxypropylene-block polymers, polyethylene glycol and wool fat, and the like.

Dosage forms that permit topical application of the compositions include ointments, pastes, creams, lotions, gels, powders, solutions, sprays, inhalants or patches. The active compound or compounds is/are mixed under sterile conditions with a pharmaceutically acceptable carrier and optionally one or more preservatives and/or buffers. The ointments, pastes, creams and gels may contain, in addition to an active compound or compounds according to the present invention, pharmaceutically acceptable carriers that permit topical or such as animal and vegetable fats, oils, waxes, paraffins, starch, tragacanth, cellulose derivatives, polyethylene glycols, silicones, bentonites, silicic acid, talc and zinc oxide, or mixtures thereof.

The pharmaceutical composition may also be a dentifrice. In the present invention, “dentifrice” is understood to broadly include compositions suitable for administering to the oral cavity, especially, for example, to the gingival/mucosal tissue or to the teeth. Thus, the dentifrice may include toothpastes, toothpowders, liquid dentifrices, mouth detergents, mouthwashes, troches, chewing gums, dental or gingival massage creams, dental strips, dental gels, and gargle tablets. When the pharmaceutical composition of this invention is a dentifrice such as tooth paste, a tooth or gum adherence promoting substance selected from the group consisting of copolymers of methyl vinyl ether and maleic anhydride, copolymers of vinyl pyrrolidone and vinyl acetate, and cyclodextrins may also be included in the composition. Copolymers of methyl vinyl ether and maleic anhydride useful in this invention may have molecular weights ranging from 200,000 to 2,000,000 kD and may be free acids, mixed sodium and calcium salts, or half ester derivatives. Copolymers of vinyl pyrrolidone and vinyl acetate useful in the invention typically have a molecule weight of approximately 27,000 kD and are water soluble. Cyclodextrins useful in the invention are cyclic oligosaccharides composed of either 6, 7 or 8 glucose units (a-, b- and g-cyclodextrin, respectively).

When the composition of this invention is a dentifrice, an antimicrobial agent is selected from the group consisting of triclosan, metronidazole, tetracyclines, quinolones, plant essential oils, camphor, thymol, carvacrol, menthol, eucalyptol, and methyl salicylate may also be included. Pharmaceutically acceptable carriers that permit administration of the compositions of this application as dentifrices include sorbitol, glycerin, silica, sodium lauryl sulfate and Xanthum gum. The dentifrices of this invention may also include sodium fluoride.

The compositions for use according to the invention can also be used as hand sanitizers or hand antiseptics. Various preparations are available, including gel, foam, and liquid solutions. Additional active ingredient in hand sanitizers may be added (e.g., volatile alcohols such as isopropanol, ethanol, or n-propanol). Inactive ingredients typically may include a thickening agent such as polyacrylic acid for alcohol gels, humectants such as glycerin for liquid rubs, propylene glycol, and essential oils of plants.

With regard to human personal care products, the present invention provides for embodiments in which the compositions further comprise an alcohol which is not an alkanediol and/or an alkanediol. In particular, non-limiting embodiments, the carbon backbone of the alkanediol has between 9 and 25 carbon atoms.

In some embodiments, the pH of a composition according to the invention for use in a human personal care product is between about 3.5-5.0, and preferably between about 4-4.7. In addition to the above ingredients, such a composition of the invention may optionally further comprise an emollient to further reduce irritation, such as, but not limited to, a fatty alcohol, behentrimonium methoslfate-cetyl alcohol, or a polyol such as glycerol, propylene glycol, diglycerol, ethylene glycol, diethylene glycol, triethylene glycol, dipropylene glycol, tripropylene glycol, hexylene glycol, butylene glycol, etc. A composition of the invention may optionally comprise a hydrophilic or hydrophobic gel forming polymer, a fatty acids, a plant oils etc. Suitable hydrophilic gel polymers include, but are not limited to, hydroxypropylmethyl cellulose, cationic hydroxyethyl cellulose, ethyl cellulose, hydroxypropyl cellulose, hydroxymethyl cellulose, carboxy methyl cellulose, polyethylene oxide (polyox resins), and chitosan pyrrolidone carboxylate, silica gel, carbomerpolymers etc. Suitable hydrophobic gel polymers include, but are not limited to, silicone polymers, for example polydimethylsiloxane polymer, dimethiconol fluid in dimethicone, cyclomethicone and dimethicone copolyl, silicone glycol, KSG series Silicone gels, and combinations thereof. Suitable plant oils include, but are not limited to, olive oil, almond oil, avocado oil, basil oil, primrose oil, peanut oil, safflower oil, sesame oil, soyabean oil, wheat germ oil.

In preferred, non-limiting embodiments, the amounts of the active agents are such that regular exposure of human skin to the personal care product does not produce skin irritation in a normal human.

Non-limiting examples of human personal care products which may utilize the invention include bar soap, liquid soap (e.g. hand soap), hand sanitizer, cleansing wipes, body wash, acne treatment products, shampoo, conditioner, cosmetics (including but not limited to liquid or powder foundation, liquid or solid eyeliner, mascara, cream eye shadow, tinted powder, “pancake” type powder to be used dry or moistened, etc.) deodorant, body lotion, hand cream, topical cream, aftershave lotion, skin toner, mouth wash, toothpaste, sunscreen lotion, and baby products such as, but not limited to, cleansing wipes, baby shampoo, baby soap, and diaper cream. The present invention may also be applied to wound care items, such as, but not limited to, wound coverings, bandages, tape, and steri-strips.

Human personal care compositions according to the invention may further comprise one or (preferably) more than one component selected from the group consisting of emollients, stabilizing agents, thickening agents, humectants, antimicrobial agents, neutralizing agents, surfactants, water, silicone polymers, alcohols, and hydrogels, as well as additional components as may be known in the art. Non-limiting examples of such components are set forth below. Suitable emollients, for example, include PEG 20 almond glycerides, Probutyl DB-10, Glucam P-20, Glucam E-10, Glucam P-10, Glucam E-20, Glucam P-20 distearate, glycerin, propylene glycol, octoxyglycerine, cetyl acetate, acetylated lanolin alcohol, cetyl ether, myristyril ether, hydroxylated milk glycerides, polyquaternium compounds, copolymers of dimethyl dialyl ammonium chloride and acrylic acid, dipropylene glycol methyl ethers, polypropylene glycol ethers and silicon polymers. Other suitable emollients may include hydrocarbon-based emollients such as petrolatum or mineral oil, fatty ester-based emollients, such as methyl, isopropyl and butyl esters of fatty acids such as isopropyl palmitate, isopropyl myristate, isopropyl isostearate, isostearyl isostearate, diisopropyl sebacate, and propylene dipelargonate, 2-ethylhexyl isononoate, 2-ethylhexyl stearate, C12-C16 fatty alcohol lactates such as cetyl lactate and lauryl lactate, isopropyl lanolate, 2-ethylhexyl salicylate, cetyl myristate, oleyl myristate, oleyl stearate, oleyl oleate, hexyl laurate, and isohexyl laurate. Additional useful emollients include lanolin, olive oil, cocoa butter, and shea butter.

Suitable thickening agents for a human personal care product may include, for example, stearyl alcohol, cationic hydroxy ethyl cellulose, hydroxy propyl methyl cellulose, hydroxy propyl cellulose, chitosan pyrrolidone carboxylate, behenyl alcohol, zinc stearate, emulsifying waxes, an addition polymer of acrylic acid, a resin, guar gum, acacia, acrylates/steareth-20 methacrylate copolymer, agar, algin, alginic acid, ammonium acrylate co-polymers, ammonium alginate, ammonium chloride, ammonium sulfate, amylopectin, attapulgite, bentonite, C9-15 alcohols, calcium acetate, calcium alginate, calcium carrageenan, calcium chloride, caprylic alcohol, carbomer 910, carbomer 934, carbomer 934P, carbomer 940, carbomer 941, carboxymethyl hydroxyethyl cellulose, carboxymethyl hydroxypropyl guar, carrageenan, cellulose, cellulose gum, cetearyl alcohol, cetyl alcohol, corn starch, damar, dextrin, dibenzlidine sorbitol, ethylene dihydrogenated tallowamide, ethylene diolamide, ethylene distearamide, gelatin, guar gum, guar hydroxypropyltrimonium chloride, hectorite, hyaluronic acid, hydrated silica, hydroxybutyl methylcellulose, hydroxyethylcellulose, hydroxyethyl ethylcellulose, isocetyl alcohol, isostearyl alcohol, karaya gum, kelp, lauryl alcohol, locust bean gum, magnesium aluminium silicate, magnesium silicate, magnesium trisilicate, methoxy PEG-22/dodecyl glycol copolymer, methylcellulose, microcrystalline cellulose, montmorillonite, myristyl alcohol, oat flour, oleyl alcohol, palm kernel alcohol, pectin, PEG-2M, PEG-5M, polyacrylic acid, polyvinyl alcohol, potassium alginate, potassium aluminium polyacrylate, potassium carrageenan, potassium chloride, potassium sulfate, potato starch, propylene glycol alginate, sodium acrylate/vinyl alcohol copolymer, sodium carboxymethyl dextran, sodium carrageenan, sodium cellulose sulfate, sodium chloride, sodium polymethacylate, sodium silicoaluminate, sodium sulfate, stearalkonium bentotnite, stearalkonium hectorite, stearyl alcohol, tallow alcohol, TEA-hydrochloride, tragacanth gum, tridecyl alcohol, tromethamine magnesium aluminium silicate, wheat flour, wheat starch, xanthan gum, abietyl alcohol, acrylinoleic acid, aluminum behenate, aluminum caprylate, aluminum dilinoleate, aluminum salts, such as distearate, and aluminum isostearates, beeswax, behenamide, butadiene/acrylonitrile copolymer, C29-70 acid, calcium behenate, calcium stearate, candelilla wax, carnauba, ceresin, cholesterol, cholesterol hydroxystearate, coconut alcohol, copal, diglyceryl stearate malate, dihydroabietyl alcohol, dimethyl lauramine oleate, dodecanoic acid/cetearyl alcohol/glycol copolymer, erucamide, ethylcellulose, glyceryl triacetyl hydroxystearate, glyceryl tri-acetyl ricinolate, glycol dibehenate, glycol di-octanoate, glycol distearate, hexanediol distearate, hydrogenated C6-14 olefin polymers, hydrogenated castor oil, hydrogenated cottonseed oil, hydrogenated lard, hydrogenated menhaden oil, hydrogenated palm kernel glycerides, hydrogenated palm kernel oil, hydrogenated palm oil, hydrogenated polyisobutene, hydrogenated soybean oil, hydrogenated tallow amide, hydrogenated tallow glyceride, hydrogenated vegetable glyceride, hydrogenated vegetable oil, Japan wax, jojoba wax, lanolin alcohol, shea butter, lauramide, methyl dehydroabietate, methyl hydrogenated rosinate, methyl rosinate, methylstyrene/vinyltoluene copolymer, microcrystalline wax, montan acid wax, montan wax, myristyleicosanol, myristyloctadecanol, octadecene/maleic anhyrdine copolymer, octyldodecyl stearoyl stearate, oleamide, oleostearine, ouricury wax, oxidized polyethylene, ozokerite, paraffin, pentaerythrityl hydrogenated rosinate, pentaerythrityl tetraoctanoate, pentaerythrityl rosinate, pentaerythrityl tetraabietate, pentaerythrityl tetrabehenate, pentaerythrityl tetraoleate, pentaerythrityl tetrastearate, ophthalmic anhydride/glycerin/glycidyl decanoate copolymer, ophthalmic/trimellitic/glycols copolymer, polybutene, polybutylene terephthalate, polydipentene, polyethylene, polyisobutene, polyisoprene, polyvinyl butyral, polyvinyl laurate, propylene glycol dicaprylate, propylene glycol dicocoate, propylene glycol diisononanoate, propylene glycol dilaurate, propylene glycol dipelargonate, propylene glycol distearate, propylene glycol diundecanoate, PVP/eiconsene copolymer, PVP/hexadecene copolymer, rice bran wax, stearlkonium bentonite, stearalkonium hectorite, stearamide, stearamide DEA-distearate, stearamide DIBA-stearate, stearamide MEA-stearate, stearone, stearyl erucamide, stearyl stearate, stearyl stearoyl stearate, synthetic beeswax, synthetic wax, trihydroxystearin, triisononanoin, triisostearin, tri-isostearyl trilinoleate, trilaurin, trilinoleic acid, trilinolein, trimyristin, triolein, tripalmitin, tristearin, zinc laurate, zinc myristate, zinc neodecanoate, zinc rosinate, and mixtures thereof.

A suitable humectant for a human personal care product, can be, for example, glycerin, 1-2-propylene glycol, dipropylene glycol, polyethylene glycol, 1,3-butylene glycol, or 1,2,6-hexanetriol.

In additional embodiments, one or more additional antimicrobial agents may be included in the composition for a human personal care product, for example, in the amount of between about 0.01 and 1.0 percent (weight/weight), where such antimicrobial agent may be selected from the group consisting of iodophors, iodine, benzoic acid, dihydroacetic acid, propionic acid, sorbic acid, methyl paraben, ethyl paraben, propyl paraben, butyl paraben, cetrimide, benzalkonium chloride, dequalinium chloride, chlorhexidine, chloroeresol, chlorxylenol, benzyl alcohol, bronopol, chlorbutanol, phenoxyethanol, phenylethyl alcohol, 2,4-dichlorobenzyl alcohol, thiomersal, clindamycin, erythromycin, benzoyl peroxide, mupirocin, bacitracin, polymyxin B, neomycin, triclosan, parachlorometaxylene, foscarnet, miconazole, fluconazole, itriconazole, ketoconazole, silver sulfadiazine, octoxyglycerine, biguanides such as, but not limited to, chlorhexidine free base, chlorhexidine palmitate, chlorhexidine diphosphanilate, chlorhexidine digluconate, chlorhexidine diacetate, chlorhexidine dihydrochioride, chlorhexidine dichloride, chlorhexidine dihydroiodide, chlorhexidine diperchlorate, chlorhexidine dinitrate, chlorhexidine sulfate, chlorhexidine sulfite, chlorhexidine thiosulfate, chlorhexidine di-acid phosphate, chlorhexidine difluorophosphate, chlorhexidine diformate, chlorhexidine dipropionate, chlorhexidine di-iodobutyrate, chlorhexidine di-n-valerate, chlorhexidine dicaproate, chlorhexidine malonate, chlorhexidine succinate, chlorhexidine malate, chlorhexidine tartrate, chlorhexidine dimonoglycolate, chlorhexidine monodiglycolate, chlorhexidine dilactate, chlorhexidine di-.alpha.-hydroxyisobutyrate, chlorhexidine diglucoheptonate, chlorhexidine di-isothionate, chlorhexidine dibenzoate, chlorhexidine dicinnamate, chlorhexidine dimandelate, chlorhexidine di-isophthalate, chlorhexidine di-2-hydroxynapthoate, chlorhexidine embonate, and parahexamethylenebiguanide (“PHMB”).

In further embodiments, a human personal care product composition for use according to the invention may be formulated as a hydrogel comprising a compound such as hydroxypropylmethyl cellulose, cationic hydroxyethyl cellulose, ethyl cellulose, hydroxypropyl cellulose, hydroxymethyl cellulose, carboxy methyl cellulose, polyethylene oxide (polyox resins), and chitosan pyrrolidone carboxylate may be formulated to include and an alcohol or a mixture of alcohols, for example, ethanol, isopropyl alcohol, n-propyl alcohol, and mixtures thereof; fatty alcohols, including, but not limited to, cetyl alcohol, myristol alcohol, stearyl alcohol, octyl alcohol, decyl alcohol and lauryl alcohol, and mixtures thereof; and hexanol.

In still another embodiments of the invention, a human personal care product or composition according to the invention may further comprise a silicone polymer, for example one or more than one polydimethylsiloxane polymer, dimethiconol fluid in dimethicone, cyclomethicone and dimethicone copolyl, and silicone glycol. In particular, non-limiting embodiments, the amount of silicone polymer is between about 0.1 and 1.0 percent (volume/volume).

The human personal care product compositions according to the invention may also include an emollient solvent such as a glycidyl ether having an alkyl chain up to and including 18 carbon molecules and ethoxylates and propoxylates thereof, a glyceryl ether having an alkyl chain up to and including 18 carbon molecules and ethoxylates and propoxylates thereof, a mono- or diglyceryl ether having an alkyl chain up to and including 18 carbon molecules and ethoxylates and propoxylates thereof, ethoxylate and propoxylate ethers, ethoxy diglycol esters, ethyl hexyl alcohol propoxylate, and propylene glycol ester ethoxylates and propoxylates.

The human personal care product compositions according to the invention may additionally comprise additives such as dyes, fragrances, pH adjusters, including basic pH adjusters such as ammonia, mono-, di- and tri-alkyl amines, mono-, di- and tri-alkanolamines, alkali metal and alkaline earth metal hydroxides (e.g., ammonia, sodium hydroxide, potassium hydroxide, lithium hydroxide, monoethanolamine, triethylamine, isopropylamine, diethanolamine and triethanolamine); acid pH adjusters such as mineral acids and polycarboxylic acids (e.g., hydrochloric acid, nitric acid, phosphoric acid, sulfuric acid, citric acid, glycolic acid, and lactic acid); vitamins such as vitamin A, vitamin E and vitamin C; polyamino acids and salts, such as ethylenediamine tetraacidic acid (EDTA), preservatives such as Germall plus and DMDM hydantoin, and sunscreens such as aminobenzoic acid, arobenzone, cinoxate, diioxybenzone, homosalate, menthyl anthranilate, octocrylene, octyl methoxycinnamate, octyl salicylate, oxybenzoate, padimate O, phenylbenzimidazole, sulfonic acid, sulisobenzone, titanium dioxide, trolamine salicylate and zinc oxide.

In some further embodiments of any of the above, the principal component by weight of the composition is water. In some embodiments, the composition according to the invention is at least 50%, 60%, 70%, 80%, 90%, 95%, 98% or 99% water by weight.

Methods of the Invention

In a second aspect, the invention provides a method of sanitizing portions or parts of a body (e.g., skin, feathers, hair, scalp, orifices, oral cavity, nails), said method comprising contacting the portion or part with a sanitizing composition according to the invention. The solution can be contacted or applied to the body by any suitable means as known to persons of ordinary skill in the art. For instance, the solution can be applied by any method that insures good contact between the surface or portion to be sanitized and the sanitizer composition. Such methods include bathing, washing, coating, brushing, dipping, immersing, wiping, misting, and spraying. These steps may be repeated to assure a thorough contacting. Once applied, after a residence time sufficient to assure the desired degree of sanitizing action (e.g., at least 1, 2 3, 4, 5, 6, 7, or 8 log fold-removal of a microbial contaminant), the solution may be physically removed from the treated surface by any means (e.g., evaporation, toweling, draining, air drying, or rinsing with water (e.g., potable water). Any combination of these steps may be performed in any order. In particular, the peracids and other ingredients used are preferably volatile and, hence, would leave little residue on the treated surface upon drying.

The residence time will vary with the concentration of the peracid (e.g. peroxyacetic acid), the 2-hydroxyorganic acid (e.g., L-(+)-lactic acid, and the surfactant (if any).

However, generally, it is contemplated that the surface to be treated may be contacted with the aqueous sanitizer composition for a residence time of from about 10 seconds to about 4 minutes. More preferably, the residence time is from about 20 seconds up to about 1 minute. The residence time can vary in accordance with the temperature and concentration of the peracid and 2-hydroxyorganic acid. Lower temperatures and concentrations would require longer contact times as could be readily empirically determined by a person of ordinary skill in the art.

The sanitizer solution can be effectively applied at comfortable temperatures suitable for liquid water. Conveniently, the temperature can be ambient or room temperature (e.g., 20° C. to 35° C.), or refrigerator temperatures 2 to 5° C. However, other temperatures can be used in accordance with the preferences or convenience of the person being treated.

In some embodiments, the contacting reduces a microbial contamination on the treated surface by at least 1, 2, 3 or 4 log units, more preferably, by at least 1 log units, and still more preferably by at least 2 log units. The contaminant can be human pathogen (e.g., E. Coli, a strain of E. coli O157H7, Listeria monocyogenes, Salmonella) or an indigenous microorganism typically found on the surface. In some embodiments, the microbial contaminant to be reduced by the treatment is a human pathogen (e.g., enterotoxic bacterium), including but not limited to, a bacterium (e.g., E.coli O157H7, Listeria moncytogenes, Salmonella), virus, a fungus, or a mold.

It has also been surprisingly found that the co-formulation of the peracid (e.g., peroxyacetic acid) with the 2-hydroxy organic acid (e.g., L-(+)- lactic acid) in the aqueous sanitizer composition provides a particularly effective and long-lasting sanitizer composition.

In some embodiments, the sanitizing composition is provided as an aqueous pre-blend mixture (e.g., about a 5-200-fold concentrate, a 5-, 10-, 20-, 40-, 50- or 100- fold concentrate) to be added to the water to be contacted with the surface to be treated. The present compositions can be employed for reducing the population of pathogenic microorganisms, such as pathogens of humans, animals, and the like. The reduced-odor compositions can exhibit activity against pathogens including fungi, molds, bacteria, spores, and viruses, for example, parvovirus, coxsackie virus, herpes virus, S. aureus, E. coli, Streplococci, Legionella, mycobacteria, or the like. Such pathogens can cause a varieties of diseases and disorders, including athletes foot, hairy hoof wart disease, mastitis or other mammalian milking diseases, tuberculosis, and the like. In addition, the present compositions can kill pathogenic microorganisms that spread through transfer by water, air, or a surface substrate.

The present methods require a certain minimal contact time of the composition with the treated surface for occurrence of significant antimicrobial effect. The contact time can vary with concentration of the use composition, method of applying the use composition, temperature of the use composition, degree of sanitizing desired, and the estimated amount of a contaminant of concern. Preferably the exposure time is at least about 5 to about 15 seconds or from 15 secs to 2 minutes. In specific non-limiting embodiments, the composition is exposed to the surface for at least 20 seconds, at least 30 seconds, or at least 60 seconds, or at least 5 minutes or at least 10 minutes. In various non-limiting embodiments, the surface may be the a skin or mucosal surface.

The following examples are intended to illustrate, but not limit, the invention.

### EXAMPLES

### Example 1

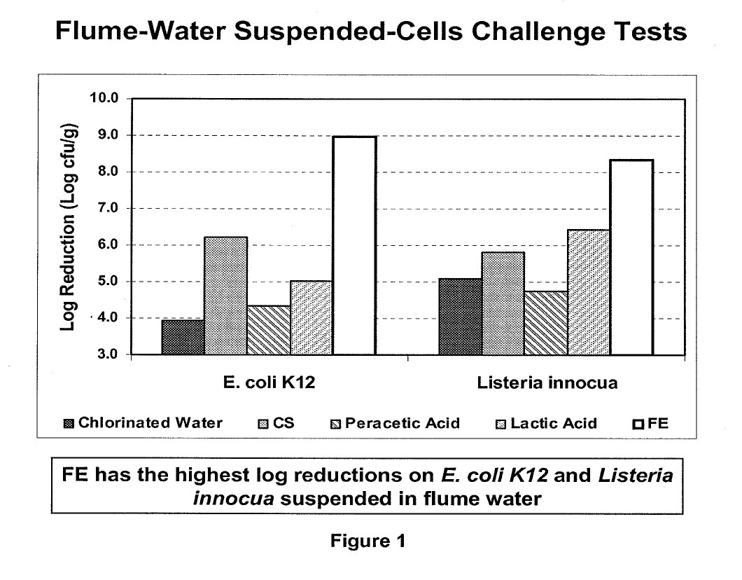

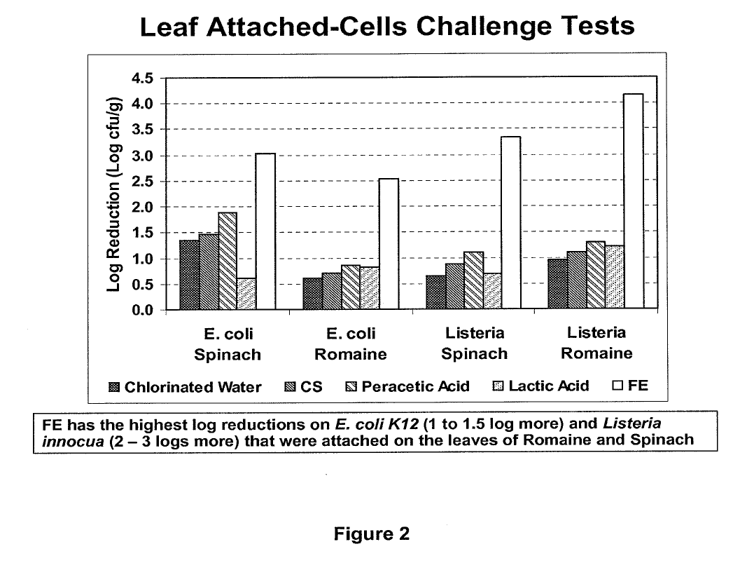

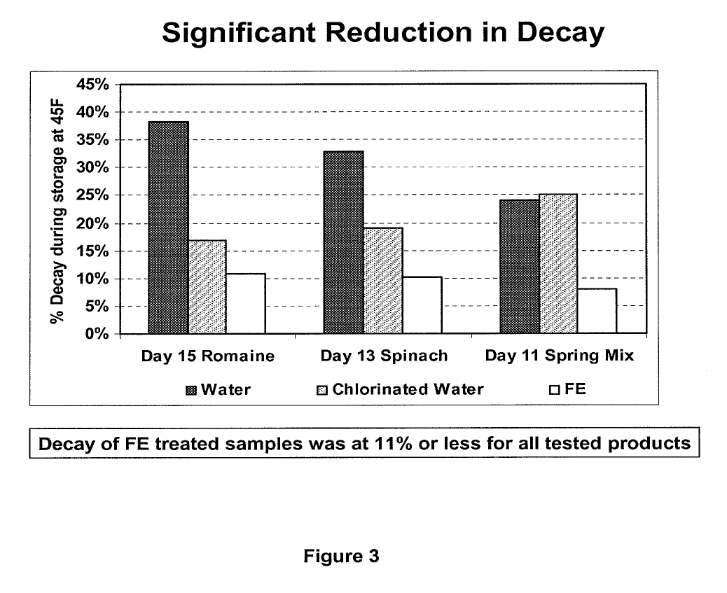

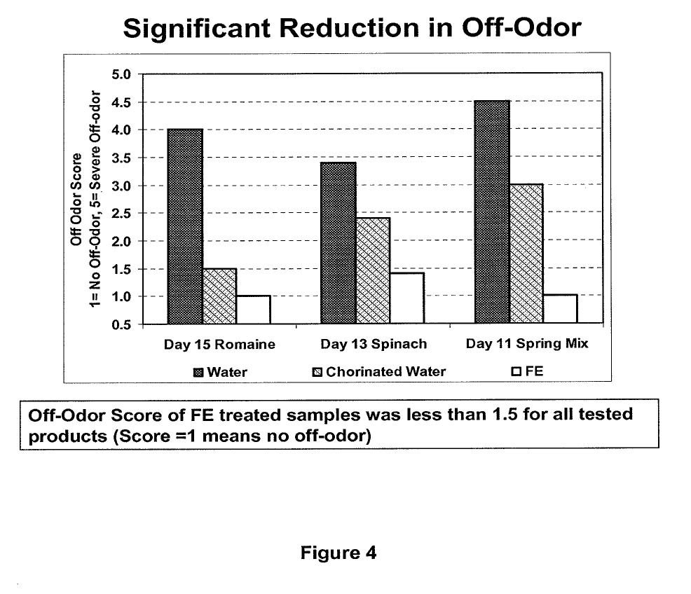

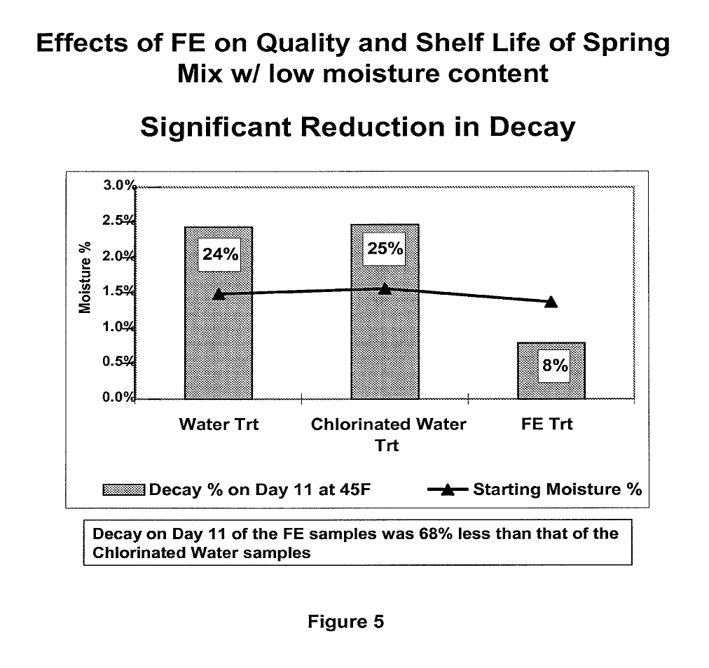

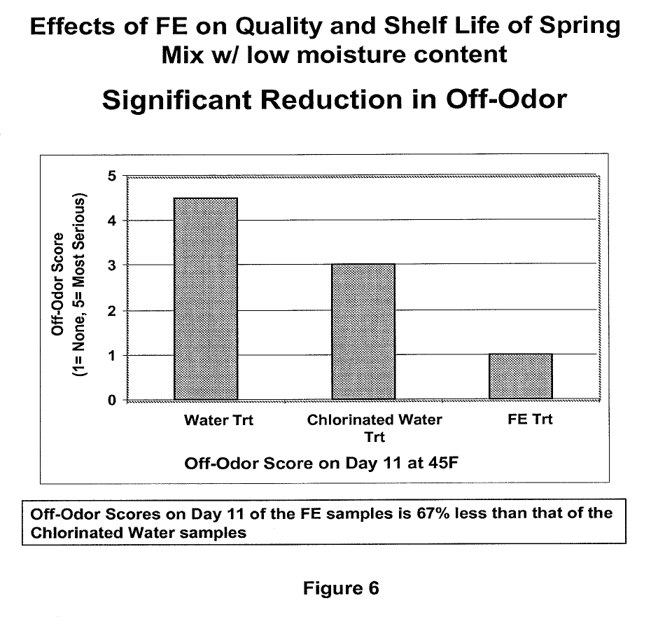

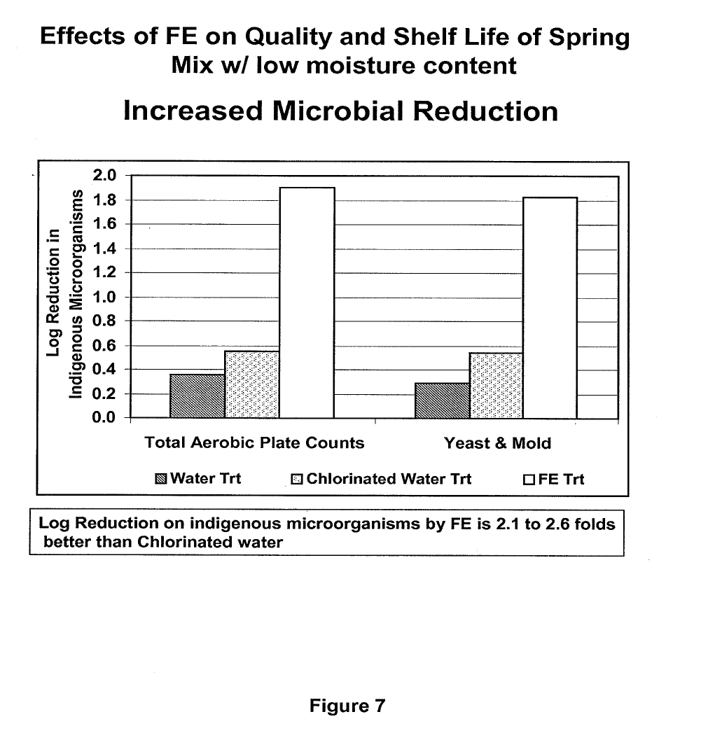

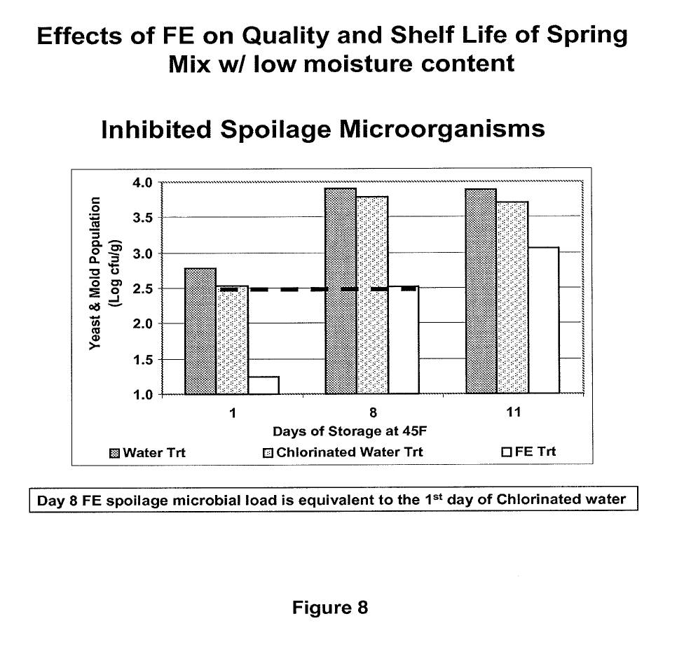

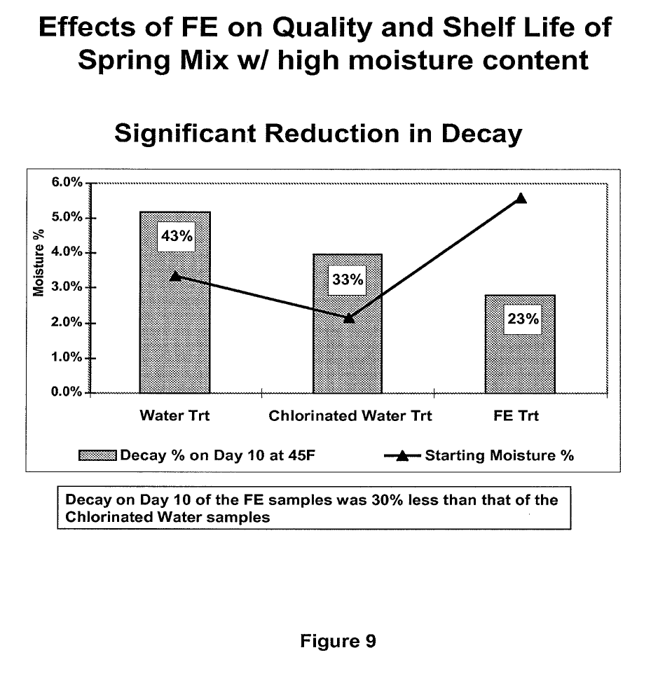

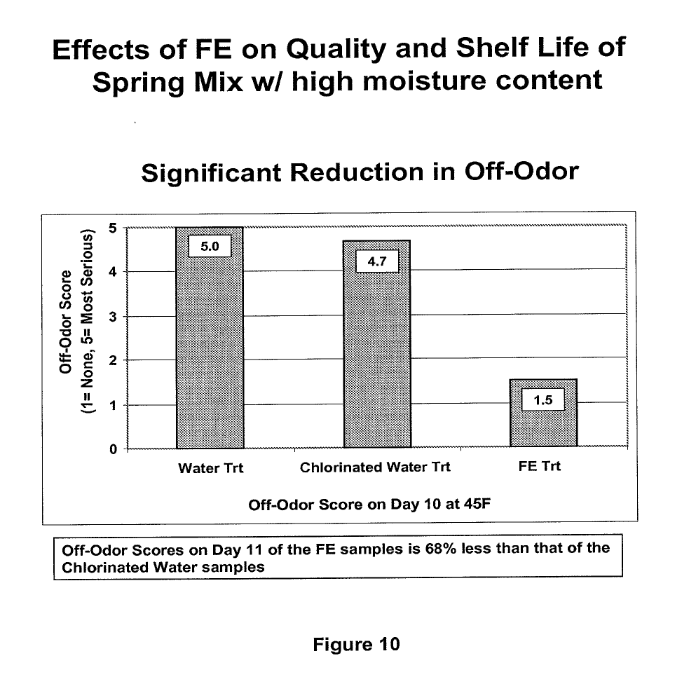

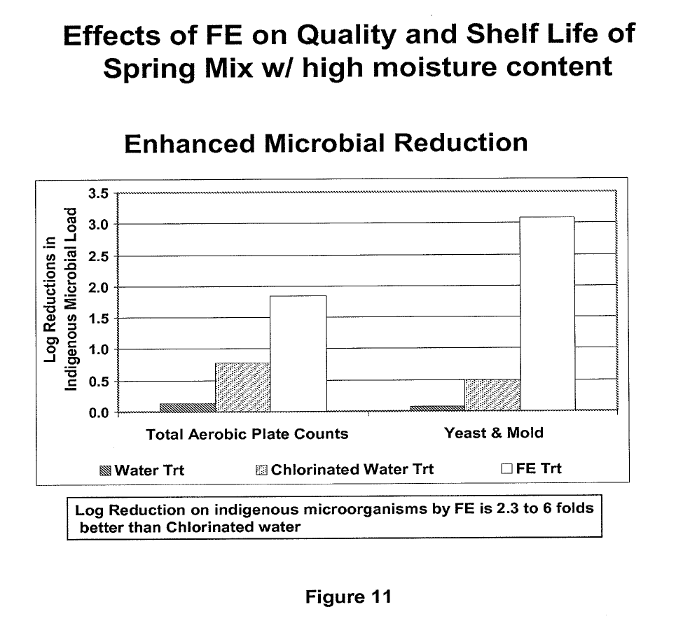

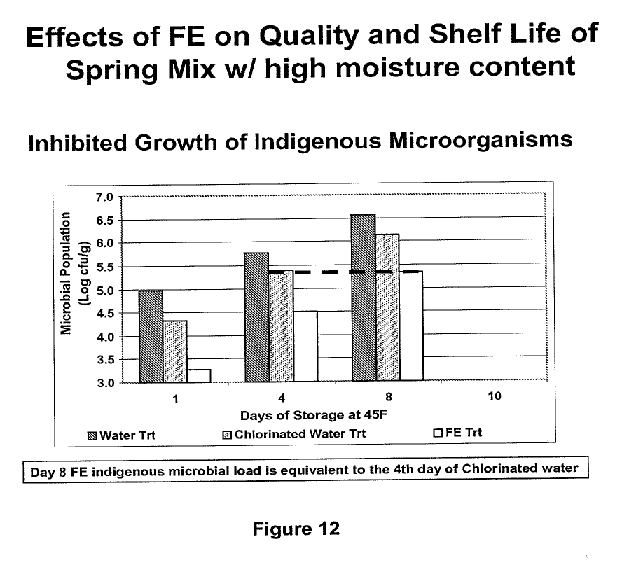

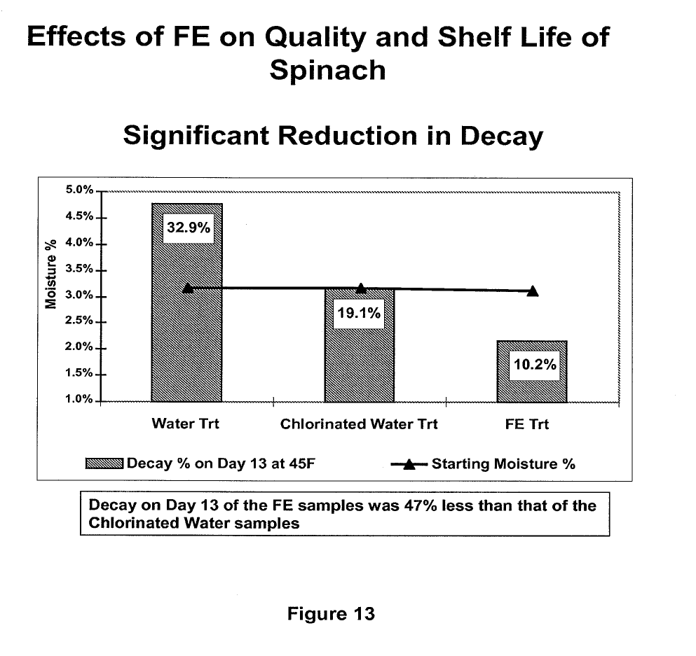

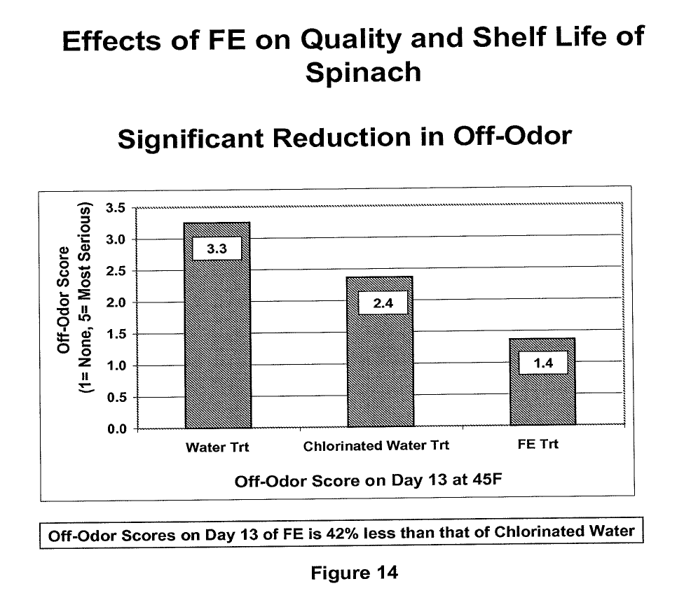

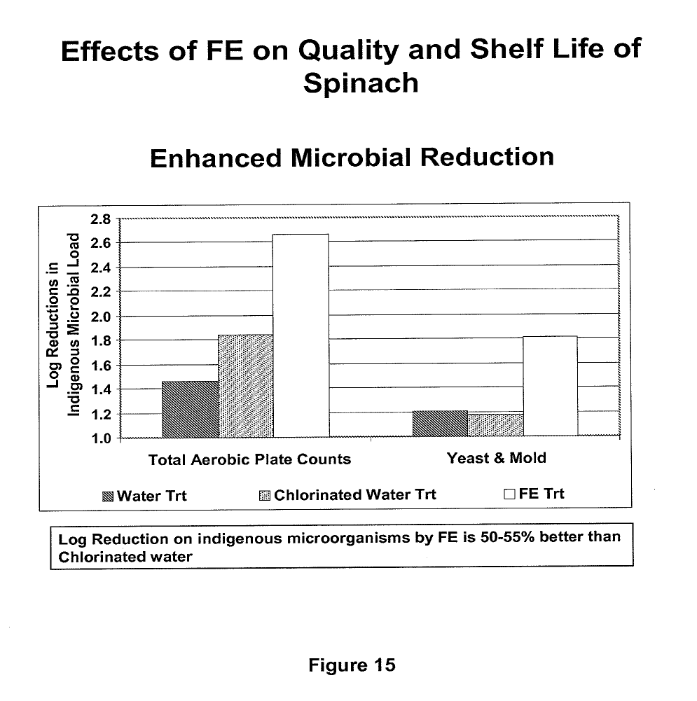

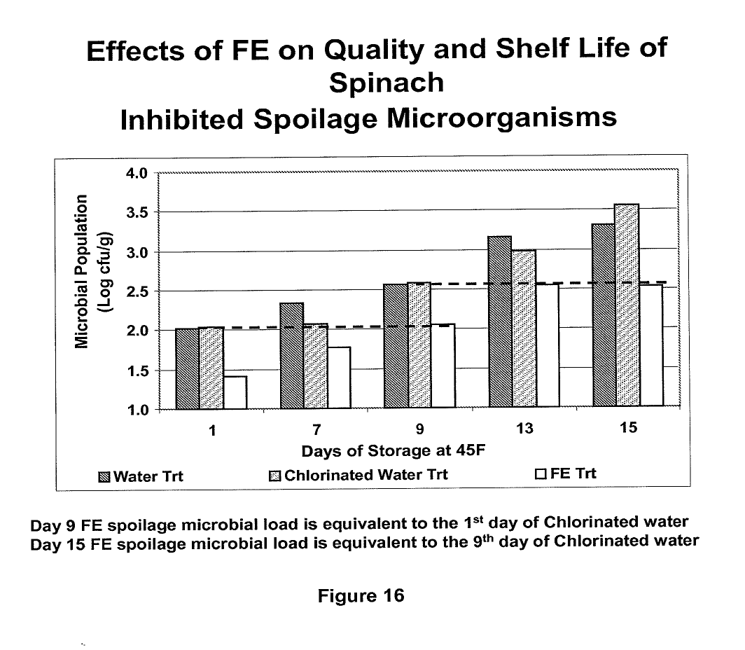

The present example illustrates the use of an aqueous sanitizing solution according to the invention. As illustrated in FIGS. 1 to 16, the solutions according to the invention advantageously remove microorganisms from the surfaces of harvested produce, inhibit the growth of indigenous microorganisms on the treated surface, and can remove model pathogens from the treated surface. The findings extend to such diverse microorganisms as bacteria, yeast, and mold.

A. Standard Operating Procedure for Shelf Life Study

This method can be used to determine the shelf life of produce that has been treated by a sanitizing solutions, generally and, particularly, those according to the invention.

Preparation

Cooled eight 20-gallon containers with 75% water to ˜45° F. Autoclave twelve 5-gallons tubs wrapped well in tin foil at least 1 day in advance of processing.
1. Depending on the type of produce, use the corresponding OTR tubes; cut, marked, and sealed to form bags. Place the bags under an UV light in the biological safety cabinet for 2 h to minimize contamination .

Processing
1. Formulate chemical sanitizers immediately before usage. All calculations are based on mass/mass.
2. Fill containers to ¾ full only so as to prevent overflowing during processing
3. Place raw product gently into a stainless steel basket with lid and fill it to ¾ full.
4. Start the timer when the basket is submerged into the chemical sanitizer
5. Cycle up and down the filled basket gently for 30 s
6. Remove the treated basket with produce from the container with chemical solution and immediately transfer it into another container ¾ filled with water for rinsing
7. Cycle up and down 10 times in water to remove the majority of residual chemical on the treated produce surface
8. Place the basket with the treated produce in an inverted manner and empty the contents gently into a dryer bin liner
9. Repeat Steps ‘3’ to ‘8’ until there the dryer bin liner is full. Closed the dryer lid and centrifuge for 20 min
10. Empty the dried produce from the bin liner to sterile tubs and let the dried treated produce sit for an extra 10-15 minutes for moisture equilibration with the environment to achieve the same moisture content as the corresponding production facility.
11. Clean all tools, equipment, and containers
12. Repeat Steps ‘1’ to ‘11’ for other sanitizer treatments

Bagging and Sealing
1. Tare the scale with the bag every time.
2. Fill the bag with the target produce mass
3. Seal bags with a proper sealing machine
4. Store in boxes at 45 F and perform evaluations: microbiological analysis,

Open Bag Evaluation (OBE), visual inspection on the appropriate days of interest.

Evaluations
1. Use the appropriate forms for OBE.
2. Visually inspect the produce and photographs the differences of the samples from various chemical
   a. OBE moisture determination- weigh initial mass of leaves, spread leaves onto folded paper towels and blot dry by pressing hands to remove exterior moisture and take a final weight.
   Calculations:
      Volume to be used of a stock solution with a concentrated solution:

$${M}_{stock} = \frac{[ Desired ] {M}_{desired}}{[ Stock ]}$$

Moisture difference:

$$Difference = ( {M}_{befire} ) - ( {M}_{after} )$$

Moisture Percentage:

$${\%}_{moisture} = \frac{( {M}_{befire} ) - ( {M}_{after} )}{( {M}_{before} )}$$

3. For visual analysis be sure that bags are labeled before first analysis to follow the same bags throughout shelf-life
4. Enumerate microbial population of the treated produce using serial dilution and spread plating.
5. Samples for microbial and OBE analysis may be retrieved, for instance, on days 1, 5, 7, 9, 12, and 15.

B. Standard Operating Procedure for Suspended Cells Challenge Test

This procedure is used to determine the antimicrobial activity of sanitizers on microorganisms that are suspended in a liquid.

Processing parameters and treatments
1. Temperature: 45 F
2. Residence time: 30+/−10 secs pH: 3+/−0.3
3. Pathogen surrogates: E. coli K12, Listeria innocua
4. Spoilage microorganisms surrogates: Pseudomonas flourescens, Saccharomyces cerevisiae

Running the test
1. Transfer 1.00 mL of a 10⁸ cfu/g stock culture into a test tube containing 9.00 gm of tested solution
2. Vortex the mixture for 15 s
3. Stop the reaction by transferring 1 mL of the treated samples to 9 mL of Butterfield Phosphate Buffer
4. Enumerate viable residual cells through serial dilutions and spread plating
5. Ensure that the operating temperature is kept at 45±1° F. (only one test tube is removed out of the fridge at a time as the kinetics of chemicals change significantly if the whole test is run at room temperature)

C. Standard Operating Procedure for Attached Cells Challenge Test

This method can be used to determine the antimicrobial activity of sanitizers on microorganisms that are attached on the surface of leaves

Processing Parameters and Treatments
1. Temperature: 45° F.
2. Residence time: 45 s
3. pH: 3+/−0.3
4. Treatments: water, chlorinated water, CS, lactic acid, peroxyacetic acid, FE sanitizer (i.e., here, aqueous solutions comprising peroxyacetic acid and lactic acid) at 16 levels
5. Products tested: Romaine, spinach, spring mix
6. Pathogen surrogate: E. coli K12, Listeria innocua
7. Microorganisms tested: indigenous microorganisms on produce leaves (Total aerobic plate counts [APC], yeast, and mold [YM])

Sample Preparation
1. Take 3-4 leaves of the tested produce and place them into a 6″×6″×5″ sterile polypropylene (PP) basket. If the tested produce is Romaine, cut the Romaine into 2″×4″ rectangles
2. Retrieve 1.00 mL of the 10⁸ cfu/g stock culture with a 1-mL pipette-man and slowly spike the leaves surface by dropping small size droplets of the innoculum onto the leaf surface. Be careful not to shake the PP basket and causes the droplets to fall out of the leaves prior to drying
3. Let the basket with the spiked leaves sit in a biological safety cabinet with a fan running (˜0.5 W.C.) for 1.75 hrs
4. Remove the PP baskets with spiked leaves from the cabinet and transfer them into a cold room/refrigerator at 40-45° F. for 0.25 hrs

Treatment of Spiked Leaves
1. Place a PP basket with spiked leaves into a sterile container containing 3-L of 45 F water for 45 seconds with swirling
2. Rinse immediately for 10 seconds by dipping the treated basket into tap water at 45 F
3. Take treated leaves from the basket and place them into a stomacher bag by means of a sterile tong
4. Label the stomacher bag with the associated treatment for the leaves
5. Repeat the Step 1 to 4 with the other treatments of the test

Enumeration of Treated Leaves
1. Add phosphate buffer into a stomacher bag with the treated leaves until a 10-fold dilution is attained
2. Stomach the bag with phosphate buffer and treated leaves for 30 seconds
3. Shake the leaves back into the phosphate buffer solution and repeat the stomaching for another 30 seconds
4. Remove buffer from stomached sample and enumerate for residual cells by serial dilution and spread plating
5. Repeat Step 1 to 4 for all other treatments

D. Standard Operating Procedure for Preparation of Microbial Stock Culture

This procedure is used to prepare a 10⁸-10⁹ cfu/mL stock culture for suspended and attached cells challenge tests. The cell concentration of the stock culture is enumerated prior to testing solution.
1. ACTIVATION OF STOCK CULTURE
   a. All procedures are done in a sterile environment (e.g. inside a Biological Safety Cabinet)
   b. A loop of cells is retrieved from the pure stock culture by means of a sterile loop. The loop of cells is aseptically transferred into a test tube with 10-mL of sterile growth medium (broth).
   c. Step “b” is repeated 3 times
   d. Incubate inoculated tubes from Step “b” and “c” for 2 days under an optimal growing temperature for the microorganism to be activated
   e. Step “b” to “d” is referred to as the first transfer (1^{st} T)
   f. Retrieve 0.1-mL of growth medium from a test tube of the 1^{st} T and aseptically transfer it into another test tube with 10-mL of sterile growth medium
   g. Verify that the tube from 1^{st} T has pure culture by spread plating a 50 to 100-uL sample of growth medium onto an agar plates
   h. Repeat Step “g” 2 times
   i. Incubate both the plates and transfer tubes #2 for two days at selected optimum temperature
   j. Steps “f” to “i” are referred to as 2^{nd} T
   k. Repeat Steps “1” to “i” with 100mL growth medium for the 3^{rd} T
   l. Store the resulted Erlenmeyer culture flasks from 3^{rd} T in refrigerator overnight
   m. Take the 3^{rd} T flask from Step “1” and transferred it equally into 4 centrifuge tubes
   n. Centrifuge the tubes with pure stock culture at 10,000 RPM for 10 minutes
   o. Decant immediately the growth medium. A pellet of cells would be formed at the bottom of the centrifuged tube
   p. Add the same amount of sterile de-ionized water to the pellet of cell
   q. Vortex to loosen and re-suspend the pellet of cells
   r. Repeat Step “n” and “o” two more times
   s. To obtain a final 10⁸-10⁹ cfu/gm of suspended cell culture, add 1/10 of the initial volume of sterile de-ionized water to the cell pellet of Step “r”
   t. Consolidate all the re-suspended cell cultures into one centrifuge tube to form the final suspended stock culture

The effects of a sanitizing solution according to the invention on the removal of microbes on the surface of produce.

Results

The following tables show the results of the suspended-cells challenge tests with and without surfactant:

| | Log Reductions | | |
|---|---|---|---|
| | PAA (ppm) | | |
| Concentration | 60 | 70 | 80 |

| | |
|---|---|
| Listeria Suspended | Surfactant No |

| | | | | |
|---|---|---|---|---|
| LA (%) | 0.6% | 3.4 | 5.0 | >8.4 |
| | 0.9% | 4.5 | 6.0 | >8.4 |
| | 1.2% | 4.9 | 6.0 | >8.4 |

| | |
|---|---|
| Listeria Suspended | Surfactant Yes |

| | | | | |
|---|---|---|---|---|
| LA (%) | 0.6% | 6.3 | 7.7 | >9.0 |
| | 0.9% | 7.7 | 7.5 | >9.0 |
| | 1.2% | 7.6 | 8.0 | >9.0 |
| Water | Control | 0.0 | | |
| Chlorine | 64 ppm | 2.1 | | |
| CS | 0.6% | 3.2 | | |

| | Log Reductions | | |
|---|---|---|---|
| | PAA (ppm) | | |
| Concentration | 60 | 70 | 80 |

| | |
|---|---|
| E. Coli Suspended | Surfactant No |

| | | | | |
|---|---|---|---|---|
| LA (%) | 0.6% | 5.6 | 6.2 | 6.6 |
| | 0.9% | 6.1 | 7.3 | 8.7 |
| | 1.2% | 7.2 | 8.5 | >9 |

| | |
|---|---|
| Listeria Suspended | Surfactant Yes |

| | | | | |
|---|---|---|---|---|
| LA (%) | 0.6% | 5.6 | 6.6 | 6.8 |
| | 0.9% | 6.2 | 8.4 | >9 |
| | 1.2% | 8.4 | 9.1 | >9 |
| Water | Control | 0.0 | | |
| Chlorine | 64 ppm | 3.7 | | |
| CS | 0.6% | 6.1 | | |

The following tables show the results for the attached-cells challenge test:

| | |
|---|---|
| | Spinach |
| E. Coli Attached | PAA (ppm) |

| | Concenration | 0 | 60 | 70 | 80 |
|---|---|---|---|---|---|
| LA (%) | 0.0% | 0.00 | 0.69 | 1.33 | 2.46 |
| | 0.6% | 0.09 | 0.65 | 1.70 | 2.94 |
| | 0.9% | 0.42 | 1.37 | 1.92 | 3.70 |
| | 1.2% | 0.81 | 1.82 | 2.37 | 4.17 |
| Chlorine | 64 ppm | 1.35 | | | |
| CS | 0.6% | 1.47 | | | |

| | |
|---|---|
| | Romaine |
| E. Coli Attached | PAA (ppm) |

| | Concenration | 0 | 60 | 70 | 80 |
|---|---|---|---|---|---|
| LA (%) | 0.0% | 0.05 | 0.26 | 0.53 | 1.18 |
| | 0.6% | 0.24 | 0.47 | 0.76 | 1.68 |
| | 0.9% | 0.37 | 1.06 | 1.39 | 2.60 |
| | 1.2% | 1.28 | 1.25 | 1.64 | 4.51 |
| Chlorine | 64 ppm | 0.61 | | | |
| CS | 0.6% | 0.71 | | | |

| | |
|---|---|
| | Spinach |
| Listeria Attached | PAA (ppm) |

| | Concenration | 0 | 60 | 70 | 80 |
|---|---|---|---|---|---|
| LA (%) | 0.0% | 0.0 | 0.3 | 0.5 | 1.2 |
| | 0.6% | 0.1 | 0.3 | 1.6 | 3.0 |
| | 0.9% | 0.2 | 0.3 | 2.0 | 3.5 |
| | 1.2% | 0.2 | 0.7 | 3.9 | 3.9 |
| Chlorine | 64 ppm | 0.4 | | | |
| CS | 0.6% | 0.5 | | | |

| | |
|---|---|
| | Romaine |
| Listeria Attached | PAA (ppm) |

| | Concenration | 0 | 60 | 70 | 80 |
|---|---|---|---|---|---|
| LA (%) | 0.0% | 0.0 | 0.6 | 1.0 | 1.7 |
| | 0.6% | 1.1 | 0.9 | 2.3 | 4.1 |
| | 0.9% | 1.4 | 1.6 | 3.2 | 4.5 |
| | 1.2% | 1.5 | 2.2 | 4.1 | 4.8 |
| Chlorine | 64 ppm | 1.0 | | | |
| CS | 0.6% | 1.2 | | | |

The above results accord with a surprisingly effective and striking increase in the removal of microorganisms and improvement of product shelf-life associated due to use of an aqueous solution according to the invention.

### Example 2

The next example demonstrates that the presence of a 2-hydroxy organic acid (e.g., lactic acid) greatly reduces the consumption of peroxyacetic acid during the treatment of produce and illustrates the use of an aqueous sanitizing solution according to the invention. As shown below, the solutions according to the invention advantageously conserve peroxyacetic acid during the removal of microorganisms from the surface of a variety of produce. The methods and compositions of the invention are also shown to greatly improve the shelf-life of the produce and greatly retard produce decay. The savings should extend to such diverse microorganisms as bacteria, yeast, and mold.

Synergism with respect to efficacy in a Suspended Cells Challenge Test at 20 s residence time with no surfactant.

The experimental treatment groups were tap water, chlorinated water, a FE sanitizer wash water (FE, FE sanitizer, a solution of peroxyacetic acid and lactic acid, as further specified in a given experiment). The experimental parameters were 40 to 45° F.; the residence time was 20 s; the pH:
water (˜7)
chlorinated water (6.5 to 7.1)
lactic acid (3.8 to 4.0)
peroxyacetic acid (6.5 to 6.8)
FE sanitizer wash water (2.7 to 3.2)
   The microbial surrogates were Listeria innocua or E. coli K-12 with a streptomycin resistance gene.

The experimental protocol was as follows:
1. Transfer 1.00 mL of a ˜10⁸cfu/g Lactobacillus plantarum (ATCC 14917) stock culture into a test tube containing 9.00 mL of treatment test solution
2. Vortex the mixture for 15 s
3. Stop the reaction by transferring 1 mL of the treated samples to 9 mL of Butterfield Phosphate Buffer
4. Enumerate viable residual cells through serial dilutions and spread plating with 1-mL transfers
5. Ensure that the operating temperature is kept at 40 to 45° F. (only one test tube is removed out of the fridge at a time as the kinetics of chemicals change significantly if the whole test is run at room temperature)
6. Repeat Steps 1 to 5 two more times
7. Repeat Steps 1 to 6 with flume water
8. Repeat Steps 1 to 6 with chlorinated water
9. Repeat Steps 1 to 8 with various levels of FE
10. Repeat Steps 1 to 8 with various levels of lactic acid
11. Repeat Steps 1 to 8 with various levels of peroxyacetic acid
12. Repeat Steps 1 to 11 with Listeria innocua (ATCC33090)

Estimation of Log Reductions
1. Log activation is a measure of the percent of microorganisms that are inactivated during the disinfection process and is defined as Log Inactivation=Log₁₀ (Nₒ/N_{T}) where Nₒ is the initial influent concentration of viable microorganisms; N_{T} is the concentration of surviving microorganisms. As M cfu/g=microbial population of stock culture; W cfu/g=microbial population in solution of “Water Treatment” and X cfu/g=microbial population in solution of “X Treatment,” the Log reduction caused by “Treatment X”=Log (w/x)

Results and Conclusions

TABLE 2.1
Comparison of log reduction of suspended Listeria innocua cells
by chlorinated wash water, lactic acid wash water, peroxyacetic acid
wash water, and FE sanitizer wash water

| Listeria innocua ATCC 33090 | | | 20 s Residence time | |
|---|---|---|---|---|
| | Peroxyacetic acid (ppm) | | | |
| Lactic Acid (ppm) | 0 | 70 | 75 | 80 |
| 0 | | 1.40 | 1.70 | 1.80 |
| 2000 | 0.08 | 3.11 | 4.09 | 5.15 |
| 2500 | 0.19 | 3.22 | 5.03 | 5.36 |
| 3000 | 0.05 | 3.49 | 5.04 | 7.15 |

| | |
|---|---|
| Chlorinated Water, ~15.5 ppm, ~pH 7 | 0.06 |

TABLE 2.2
Comparison of log reduction of suspended Lactobacillus plantarum
cells by chlorinated wash water, lactic acid (LA) wash water,
peroxyacetic acid (PA) wash water, and FE sanitizer wash water.

| Lactobacillus plantarum 14917 | | | 20 s Residence time | |
|---|---|---|---|---|
| | Peroxyacetic acid (ppm) | | | |
| Lactic Acid (ppm) | 0 | 70 | 75 | 80 |
| 0 | | 4.52 | 5.59 | 5.59 |
| 2000 | 0.00 | 7.09 | >7.74 | >7.74 |
| 2500 | 0.02 | 7.09 | >7.74 | >7.74 |
| 3000 | 0.01 | >7.74 | >7.74 | >7.74 |

| | |
|---|---|
| Chlorinated Water, ~15.5 ppm, ~pH 7 | 0.00 |

Log reduction of the test FE sanitizer (here, a combination of lactic acid and peroxyacetic acid as specified above) on L. innocua and L. plantarum was significantly better than PA wash water and LA wash water. This clearly indicated the synergistic effects of combining LA and PA. FE sanitizer wash water with 70 ppm PA and 2000 ppm LA at 20 s residence time provided ˜3-log₁₀ reduction on Listeria innocua. The log reduction of provided by the combination of lactic acid and peroxyacetic acid) was about significantly 2 to 4 folds better than peroxyacetic acid with no lactic acid addition.

### Example 3

The next experiments compares the effects of sanitizers on vegetative pathogens suspended in a liquid.

Processing Parameters and Treatments
Treatments: tap water, chlorinated water, FE sanitizer wash water;
Temperature: 40 to 45° F.; Residence time: 30 s
pH:
   water (˜7)
   chlorinated water (6.5 to 7.1)
   FE sanitizer wash water (2.7 to 3.2)
Pathogens:
   5-strains cocktail of E. coli O0157:H7 (F4546, F4637, SEA13B88, TW14359, 960218)
5-strains cocktail of Listeria monocytogenes (ATCC 19115, ATCC51414, ATCC15313, FRR B2472 (SCOTT A), 1838)
5-strains cocktail of Salmonella (S. Newport, S. Tennessee, S. muenchen, S cubana, S. St. Paul)

Activation of Stock Culture
1. Activation of stock culture is attained via a series of transfers of stock culture to optimum growth medium aseptically in a biological safety cabinet
2. Retrieve a small loop (˜100 uL) of pure culture from the stock culture in storage and transfer it into a test tube containing 10 mL of optimum growth medium broth specific for each microorganism as recommended by American Type Culture Collection (ATCC) or published articles
3. Incubate culture till it reaches end of log growth phase at its optimum growth temperature as recommended by ATCC or published articles
4. Verify purity of the transferred culture by streak plating and spread plating
5. Retrieve 1.5-ml of culture broth from Step 3 and transfer it into a 250-mL Erlenmeyer Flask containing 150-mL optimum growth medium broth specific for each microorganism as recommended by American Type Culture Collection (ATCC) or published articles
6. Incubate culture till it reaches end of log growth phase at its optimum growth temperature as recommended by ATCC or published articles
7. Verify purity of the transferred culture by streak plating
8. Enumerate the concentration of the culture broth from Step 6 by spread plating and serial dilution at 1-mL transfers
9. Cool down the 150-Ml Erlenmeyer Flask stock culture at refrigeration temperature for 1 to 4 h prior to inoculation

Innoculum Preparation and Enumeration
1. Separate the 150-mL of cooled-down stock culture in the 2^{nd} transfer Erlenmeyer flask into three 50-mL centrifuge tubes at equal volume (50 mL each)
2. Centrifuge the tubes at 10,000 RPM for 15 minutes at 4° C.
3. Decant the liquid broth from each centrifuge tube leaving behind the pellet of cells
4. Fill the centrifuge tube from Step 3 with 5-mL of sterile 0.1% peptone water and vortex to loosen and mix the pellet of cells
5. Pour all the re-suspended stock culture into one centrifuge tube to form a ˜10⁸ cfu/gm of innoculum
   Enumerate and confirm the microbial population of the innoculum obtained from Step ‘5’ by spread plating via serial dilutions with 1-mL transfers

Methods
6. Transfer 1.00 mL of a ˜10⁸ cfu/g E. coli 0157:H7 5-strains cocktail stock culture into a test tube containing 9.00 mL of test solution
7. Vortex the mixture for 15 s
8. Stop the reaction by transferring 1 mL of the treated samples to 9 mL of Butterfield Phosphate Buffer
9. Enumerate viable residual cells through serial dilutions and spread plating with 1-mL transfers
10. Ensure that the operating temperature is kept at 40 to 45° F. (only one test tube is removed out of the fridge at a time as the kinetics of chemicals change significantly if the whole test is run at room temperature)
11. Repeat Steps 1 to 5 two more times
12. Repeat Steps 1 to 6 with flume water
13. Repeat Steps 1 to 6 with chlorinated water (10 ppm active chlorine at pH 6.5 to 7)
14. Repeat Steps 1 to 8 with another level of FE
15. Repeat Steps 1 to 8 with another 5-strains cocktail of Listeria monocytogenes
16. Repeat Steps 1 to 8 with another 5-strains cocktail of Salmonella

Results and Conclusion

TABLE 3.1
Comparison of Log reduction of suspended E. coli O157:H7
cells by chlorinated wash water and the test FE sanitizers wash waters.

| | Microbial | |
|---|---|---|
| | population | |
| 5-Strains cocktail of E. coli O157:H7 | (log cfu/mL) | Log Reduction |
| Residence time | 30 s | |
| Test Date | Jan. 21, 2009 | |
| Temperature | 40 to 45 F. | |
| Inoculum microbial population | 9.0 | |
| Tap Water | 8.0 | |
| (9 mL water with 1 mL of inoculum) | | |
| Chlorinated Water, 10 ppm at pH 7.1 | 7.0 | 0.9 |
| (9 mL chorinated water with | | |
| 1 mL of inoculum) | | |
| FE1-PA: 68 ppm, LA; 4600 ppm, | <1.0 | >7 |
| pH 2.8 to 3 (9 mL FE sanitizer with | No residual | |
| 1 mL of inoculum) | cells at 10¹ | |
| FE2-PA: 71 ppm, LA 5100 ppm, | <1.0 | >7 |
| pH 2.8 to 3 (9 mL FE sanitizer with | No residual | |
| 1 mL of inoculum) | cells at 10¹ | |

TABLE 3.2
Comparison of Log reduction of suspended Salmonella
cells by chlorinated wash water and the test FE sanitizers wash water.

| | Microbial | |
|---|---|---|
| | population | |
| 5-Strains cocktail of Salmonella | (log cfu/mL) | Log Reduction |
| Residence time | 30 s | |
| Test Date | Jan. 21, 2009 | |
| Temperature | 40 to 45 F. | |
| Inoculum microbial population | 8.9 | |
| Tap Water | 8.0 | |
| (9 mL water with 1 mL of inoculum) | | |
| Chlorinated Water, 10 ppm | 7.0 | 1.0 |
| at pH 7.1 (9 mL chorinated | | |
| water with 1 mL of inoculum) | | |
| FE1-PA: 68 ppm, LA; 4600 ppm, | <1.0 | >7 |
| pH 2.8 to 3 (9 mL FE sanitizer | No residual | |
| with 1 mL of inoculum) | cells at 10¹ | |
| FE2-PA: 71 ppm, LA 5100 ppm, | <1.0 | >7 |
| pH 2.8 to 3 (9 mL FE sanitizer | No residual | |
| with 1 mL of inoculum) | cells at 10¹ | |

TABLE 3.3
Comparison of Log reduction of suspended Listeria monocytogenes
cells by chlorinated wash water and the test FE sanitizers wash water.

| | Microbial | |
|---|---|---|
| 5-Strains cocktail of | population | |
| Listeria moncytogenes | (log cfu/mL) | Log Reduction |
| Residence time | 30 s | |
| Test Date | Jan. 21, 2009 | |
| Temperature | 40 to 45 F. | |
| Inoculum microbial population | 7.1 | |
| Tap Water | 6.2 | |
| (9 mL water with 1 mL of inoculum) | | |
| Chlorinated Water, 10 ppm | 5.0 | 1.2 |
| at pH 7.1 (9 mL chorinated water | | |
| with 1 mL of inoculum) | | |
| FE1-PA: 68 ppm, LA; 4600 ppm, | No residual | >5.2 |
| pH 2.8 to 3 (9 mL FE sanitizer | cells at 10¹ | |
| with 1 mL of inoculum) | | |
| FE2-PA: 71 ppm, LA 5100 ppm, | No residual | >5.2 |
| pH 2.8 to 3 (9 mL FE sanitizer | cells at 10¹ | |
| with 1 mL of inoculum) | | |

10 ppm chlorinated water reduced the populations of each pathogen by ˜1-log₁₀ when compared to the tap water control. The two concentrations of FE sanitizer wash water plate counts had no residual colonies and the results were recorded as <1.0 log₁₀ cfu/mL. Hence FE sanitizer wash water delivered reductions of greater than 7-log₁₀ for E. coli O157:H7 and Salmonella, and greater than 5.2-log₁₀ for Listeria monocytogenes when compared to the tap water control. The lower reduction observed in Listeria monocytogenes does not indicate that the FE sanitizer was less effective against that pathogen as the reported results were restricted by the original population of the stock inoculum.

### Example 4

The purpose of these experiments was to determine the antimicrobial activity of sanitizers on vegetative pathogens that are attached on the surface of leaves

Processing Parameters and Treatments

Treatments: tap water, chlorinated water, test FE sanitizer wash water;

Temperature: 40 to 45° F.; Residence time: 30 s;

pH:
water (˜7)
chlorinated water (6.5 to 7.1)
FE sanitizer wash water (2.7 to 3.2)

Products tested: diced Romaine leaves and matured spinach leaves

Pathogens:
5-strains cocktail of E. coli O157:H7 (F4546, F4637, SEA13B88, TW14359, 960218)
5-strains cocktail of Listeria monocytogenes (ATCC 19115, ATCC51414, ATCC15313, FRR B2472 (SCOTT A), 1838)
5-strains cocktail of Salmonella (S. Newport, S. Tennessee, S. muenchen, S cubana, S. St. Paul)

Activation of Stock Culture
1. Activation of stock culture is attained via a series of transfers of stock culture to optimum growth medium aseptically in a biological safety cabinet.
2. Retrieve a small loop (˜100 uL) of pure culture from the stock culture in storage and transfer it into a test tube containing 10 mL of optimum growth medium broth specific for each microorganism as recommended by American Type Culture Collection (ATCC) or published articles.
3. Incubate culture till it reaches end of log growth phase at its optimum growth temperature as recommended by ATCC or published articles.
4. Verify purity of the transferred culture by streak plating and spread plating.
5. Retrieve 1.5-ml of culture broth from Step 3 and transfer it into a 250-mL Erlenmeyer Flask containing 150-mL optimum growth medium broth specific for each microorganism as recommended by American Type Culture Collection (ATCC) or published articles
6. Incubate culture till it reaches end of log growth phase at its optimum growth temperature as recommended by ATCC or published articles.
7. Verify purity of the transferred culture by streak plating.
8. Enumerate the concentration of the culture broth from Step 6 by spread plating and serial dilution at 1-mL transfers.
9. Cool down the 150-Ml Erlenmeyer Flask stock culture at refrigeration temperature for 1 to 4 h prior to inoculation.

Innoculum Preparation and Enumeration
1. Separate the 150-mL of cooled-down stock culture in the 2nd transfer Erlenmeyer flask into three 50-mL centrifuge tubes at equal volume (50 mL each).
2. Centrifuge the tubes at 10,000 RPM for 15 minutes at 4° C.
3. Decant the liquid broth from each centrifuge tube leaving behind the pellet of cells
4. Fill the centrifuge tube from Step 3 with 5-mL of sterile 5% Horse Serum solution and vortex to loosen and mix the pellet of cells.
5. Pour all the re-suspended stock culture into one centrifuge tube to form a ˜108 cfu/gm of innoculum.
6. Enumerate and confirm the microbial population of the innoculum obtained from Step ‘5’ by spread plating via serial dilutions with 1-mL transfers

Samples Preparation
1. Take 4 leaves of the tested produce and place them into a 6″×6″×5″ sterile polypropylene (PP) basket. If the tested produce is Romaine, cut the Romaine into 1.5″×2.5″ rectangles
2. Of the four leaves in Step 1, two should have their upper epidermis facing upward and two should have their lower epidermis facing upward
3. Retrieve 50 uL of the ˜10⁸ cfu/g stock culture with a 100 uL pipette and slowly spike each leaf by dropping small size droplets (10 to 15 droplets) of the inoculum onto the leaf flat surface and midrib that are facing upward. Be sure to remove excess stock on sides of pipette tip before spiking leaves. Be careful not to shake the PP basket and causes the droplets to fall out of the leaves prior to drying.
4. Arrange the baskets with the spiked leaves in a biological safety cabinet with Drierite as shown in Photo 1 for 1-1.5 hrs at 70-80 F and 38 to 48% relative humidity. Ensure that the hood temperature is steady (<±2 F) throughout the drying process.
5. Ensure that the leaves are not in wilted condition at the end of the drying period.

Treatment of Spiked Leaves

Transfer 3 L of test solution from the PP carboy into the 5-L sterile PP tub
1. Add the required volume of the final ingredient into the 3 L solution and mix thoroughly with a sterilized tong if needed
2. Transfer two spiked leaves (1 spiked on the upper epidermis and the other spiked on the lower epidermis) into an empty sterile PP basket
3. Place the PP basket with spiked leaves into a sterile container containing 3 L of the completed formulation of the test solution
4. Maintain the temperature of the test solution at 40-45° F.
5. Use a tong to gently pushed the leaves into the test solution to ensure total submersion of the leaves at all times and to prevent folding and overlapping of leaves
6. Start stop watch for timing the 30 s once the leaves are totally submerged
7. Take treated leaves from the basket and place them into a stomacher bag by means of a sterile tong
8. Label the stomacher bag with the associated treatment for the leaves
9. Smashed the leaves into pieces by means of a sanitized rubber melon hammer
10. Repeat Step 1 to 7 with the other treatments of the test
11. Each treatment must be done in triplicates following the sequence of Step 13
12. Each replicate must be performed separately to avoid error from bacterial death during the drying process. The order of testing is as followed:
   a. 1^{st} Replication: 1 sample of control with no spike, control with spiked bacteria, spiked bacteria with water wash, spiked bacteria with chlorinated water wash, spiked bacteria with FE1 wash, and spiked bacteria with FE2 wash.
   b. 2^{nd} Replication: 1 sample of control with no spike, control with spiked bacteria, spiked bacteria with water wash, spiked bacteria with chlorinated water wash, spiked bacteria with FE1 wash, and spiked bacteria with FE2 wash.
   c. 3^{rd} Replication: 1 sample of control with no spike, control with spiked bacteria, spiked bacteria with water wash, spiked bacteria with chlorinated water wash, spiked bacteria with FE1 wash, and spiked bacteria with FE2 wash.
13. Enumeration of samples must be performed immediately after each replication

Enumeration of Treated Leaves
1. Add 100 mL phosphate buffer into a stomacher bag with the treated mashed leaves until a 100-fold dilution is attained
2. Stomach the bag with phosphate buffer and treated leaves for 30 s
3. Shake the leaves back into the phosphate buffer solution and repeat the stomaching for another 30 seconds
4. Remove buffer from stomached sample and enumerate for residual cells by serial dilution and spread plating with 1-mL transfers
5. Repeat Step 1 to 4 for all other treatments

Estimation of Log Reductions
M cfu/g=microbial population on leaves without any treatment;
R cfu/g=microbial population in water solution for the “Water Treatment”;
W cfu/g=microbial population on leaves from “Water Treatment”;
X cfu/g=microbial population on leaves from “X Treatment”; Hence, Log reduction caused by “Treatment X”=Log (w/x)
Microorganisms removed due to mechanical washing=R
Microorganisms died during the drying process=M−W−R

Results

TABLE 4.1
Log reduction of pathogens attached on spinach and Romaine lettuce
(average of 3 replicates) by tap water at 40 to 45° F.

| | Tap Water Wash |
|---|---|
| E. coli O157:H7 on Spinach | 0.8 |
| E. coli O157:H7 on Romaine | 1.5 |
| Salmonella on Spinach | 0.9 |
| Salmonella on Romaine | 0.3 |
| L. monocytogenes on Spinach | 1.4 |
| L. monocytogenes on Romaine | 1.4 |

The tap water wash removed 0.3 to 1.5 log₁₀ of inoculated cells from the leaves indicating that complete attachment of cells on the leaves was not achieved. This was probably caused by the desiccation and wilting of the leaves under low relative humidity of the environment (20 to 23% rather than 38 to 48% as listed in the protocol).

TABLE 4.2
Additional log reduction of pathogens attached on spinach and
Romaine lettuce (average of 3 replicates) by chlorinated wash water
when compared with tap water wash

| | Chorinated water wash | | |
|---|---|---|---|
| | water at 40-45 F. | | |
| | | Concentration | |
| | pH | ppm | Log Reduction |
| E. coli O157:H7 on Spinach | 7.1 | 9.7 | 2.3 |
| E. coli O157:H7 on Romaine | 7.0 | 9.7 | 1.4 |
| Salmonella on Spinach | 6.9 | 9.3 | 1.2 |
| Salmonella on Romaine | 6.9 | 9.7 | 0.8 |
| L. monocytogenes on Spinach | 6.9 | 9.3 | 0.1 |
| L. monocytogenes on Romaine | 6.9 | 9.0 | 0.4 |

The 10 ppm chlorinated water provided an additional reduction of 0.1-log₁₀ to 1.4-log₁₀ on the pathogens. The 2.3-log₁₀ in the case of spinach was exceptionally high when compared with surrogate attached cells results and was probably caused by the incomplete attachment of the cells on the leaves as shown by the tap water wash results.

TABLE 4.3
Additional log reduction of pathogens attached on spinach and
Romaine lettuce (average of 3 replicates) by FE sanitizer wash
water at 40 to 45 F.

| | FE sanitizer wash | | |
|---|---|---|---|
| | water at 40-45 F. | | |
| | Peroxyacetic | Lactic | |
| | acid | acid conc | Log |
| | conc. (ppm) | (ppm) | Reduction |
| E. coli O157:H7 on Spinach | 68 | 4846 | 2.9 |
| E. coli O157:H7 on Romaine | 67 | 4800 | 2.6 |
| Salmonella on Spinach | 66 | 4833 | 2.3 |
| Salmonella on Romaine | 69 | 4758 | 2.1 |
| L. monocytogenes on Spinach | 70 | 4782 | 2.2 |
| L. monocytogenes on Romaine | 71 | 4769 | 3.4 |

The test FE sanitizer wash water (69 ppm peroxyacetic acid and 4800 ppm lactic acid) provided an additional reduction of 2.1-log₁₀ to 3.4-log₁₀ on the pathogens when compared with tap water wash.

When compared to chlorinated water, the FE sanitizer provided an additional 2-log₁₀ reduction of pathogens that were attached on leaves. In addition, storing the spread plates at 40 F indicated that injured cells were not able to grow at refrigerated temperatures within a week. If the bacterial cells were not able to grown on nutrient rich agar plates, they will most likely not grow on the treated fresh produce.

### Example 5

These experiments evaluated the consumption or depletion of peroxyacetic acid when used to wash produce. The objective accordingly was to compare the amount of chopped Romaine Lettuce required to deplete 600 gallons of chlorinated wash water, 600 gallons of peroxyacetic acid wash water, and 600 gallons of FE sanitizer wash water

### Processing Parameters and Treatments

Treatments: chlorinated water, peroxyacetic acid wash water, and FE sanitizer wash water

Temperature: 38 to 40° F.

Residence time: 20 s

pH:
chlorinated water (6.5 to 7.1)
peroxyacetic acid (6.5 to 6.8)
FE sanitizer wash water (2.7 to 3.2)

Produce: 1.5″×2″ diced Romaine lettuce

A. Determination of the amount of Romaine Lettuce that could deplete 600 gallons of peroxyacetic acid wash water.
1. Perform full sanitization on the Pilot Line System.
2. Fill the 2^{nd} flume tank, 2″ reservoir, and 2″ filtering tank with tap water.
3. Recycle the water through the system until the water in the system is being cooled down to 40° F.
4. Calibrate the Prominent System and use the Prominent System to monitor the concentration of PAA in the wash water.
5. Add the PAA to the 2^{nd} filtering tank until the target processing limit is reached.
6. Dice the Romaine Lettuce via the translicer.
7. Collect the 2″33 2″ diced Romaine in totes.
8. Record the weight of each tote prior to transferring it to the 2^{nd} flume.
9. Collect three untreated bags of Romaine Lettuce from each bin (1 top, 1 middle, and 1 bottom).
10. Collect three treated bags of Romaine Lettuce at the end of F2 (1 beginning, 1 middle, and 1 end of the bin).
11. Place white totes at the bottom of the locations with water spill. Return the spilt water back into the flume tank as needed.
12. Place white totes at the bottom outlets of the centrifuge to collect liquid that would be spin off from the leaves. Return the collected water back into the flume tank as needed.
13. Repeat Steps ‘e’ to ‘k’ for the rest of the bins till the FE concentrations fall below the lowest processing limits.
14. Enumerate the microbial population (APC and Yeast and mold) on the collected samples.

B. Determination of the Amount of Romaine Lettuce that Could Deplete 600 gallons of FE Wash Water
1. Perform full sanitization on the Pilot Line System.
2. Fill the flume tank, 2″ flume tank, 1^{st} reservoir, 2^{nd} reservoir, 1^{st} filtering tank, and 2nd filtering tank with tap water.
3. Recycle the water through the system until the water in the system is being cooled down to 40° F.
4. Switch on the by-passes for the 1^{st} and 2″ flume tank systems so that water would not be going through the filtering systems but only recycling from the flume tank to its associate reservoir continuously.
5. Add the chemical ingredients to both tank until the target processing limit is reached.
6. Verify the concentration of FE by the probe of the Prominent Monitoring System at the 1^{st} flume tank (F1), 1^{St} Reservoir (R1), 2^{nd} Flume tank (F2), and the 2^{nd} Reservoir (R2).
7. Collect water samples from F1 and F2.
8. Assemble the Romaine Lettuce Bins next to the dumpster.
9. Transfer whole Romaine Lettuce leaves from the bin to the conveyor.
10. Ensure that the lid above the F1 is closed. Turn the “ON/OFF” switch of the translicer to “ON”.
11. Turn the conveyor for transferring leaves into the translicer to “ON”.
12. Ensure that the chopped Romaine are delivered evenly into the flume tank without aggregation and clumping.
13. Collect three untreated bags of Romaine Lettuce from each bin (1 top, 1 middle, and 1 bottom).
14. Collect three treated bags of Romaine Lettuce at the end of F2 (1 beginning, 1 middle, and 1 end of the bin).
15. Verify the pH, temperature, and the concentration of FE at the 1^{st} flume tank (F1), 1^{st Reservoir (R}1), 2^{nd} Flume tank (F2), and the 2^{nd} Reservoir (R2) before and after processing a bin.
16. Place white totes at the bottom of the locations with water spill. Return the spilt water back into the flume tank as needed.
17. Place white totes at the bottom outlets of the centrifuge to collect liquid that would be spin off from the leaves. Return the collected water back into the flume tank as needed.
18. Repeat Steps ‘e’ to ‘o’ for the rest of the bins till the FE concentrations fall below the lowest processing limits.
19. Enumerate the microbial population (APC and Yeast and mold) on the collected samples.

c. Determination of the Amount of Romaine Lettuce that Could deplete 600 Gallons of Chlorinated Water to Concentration Below the Optimum
1. Perform full sanitization on the Pilot Line System.
2. Fill the 1^{st} flume tank, 2^{nd} flume tank, 1^{st} reservoir, 2^{nd} reservoir, 1^{st} filtering tank, and 2^{nd} filtering tank with tap water.

3. Recycle the water through the system until the water in the system is being cooled down to 40° F.
4. Switch on the by-passes for the 1^{st} and 2^{nd} flume tank systems so that water would not be going through the filtering systems but only recycling from the flume tank to its associate reservoir continuously.
5. Add the chemical ingredients to both tank until the target processing limit is reached
6. Verify the concentration of chlorinated water by the probe of the HACH System at the 1^{st} flume tank (F1), 1^{st} Reservoir (R1), 2^{nd} Flume tank (F2), and the 2^{nd} Reservoir (R2)
7. Collect water samples from F1 and F2.
8. Assemble the Romaine Lettuce Bins next to the dumpster.
9. Transfer Romaine Lettuce leaves from the bin to the conveyor.
10. Ensure that the lid above the F1 is closed. Turn the “ON/OFF” switch of the translicer to “ON”.
11. Turn the conveyor for tranferring leaves into the translicer to “ON”.
12. Ensure that the chopped Romaine are delivered evenly into the flume tank without aggregation and clumping.
13. Collect three untreated bags of Romaine Lettuce from each bin (1 top, 1 middle, and 1 bottom).
14. Collect three treated bags of Romaine Lettuce at the end of F2 (1 beginning, 1 middle, and 1 end of the bin).
15. Verify the pH, temperature, and the concentration of chlorinated water at the 1^{st} flume tank (F1), 1^{st} Reservoir (R1), 2^{nd} Flume tank (F2), and the 2^{nd} Reservoir (R2) before and after processing a bin.
16. Place white totes at the bottom of the locations with water spill. Return the spilt water back into the flume tank as needed.
17. Place white totes at the bottom outlets of the centrifuge to collect liquid that would be spin off from the leaves. Return the collected water back into the flume tank as needed.
18. Enumerate the microbial population (APC and Yeast and mold) on the collected samples.

Results and Conclusions

TABLE 5.1
Depletion of Peroxyacetic acid/PA with no Lactic acid/LA in the
presence of organic matter based on commercial scale test.
Product: Diced Romaine Lettuce
Volume of sanitizer 600 gallons
Wash water Temp 38 to 40 F.

| Wt. of Diced | Cumulative Wt. | | | |
|---|---|---|---|---|
| Romaine | of Diced | | | Peroxide |
| added (lb) | Romaine added (lb) | PA (ppm) | LA (ppm) | (ppm) |
| 0.0 | 0.0 | 84.8 | 0 | 7.5 |
| 55.2 | 55.2 | 83.3 | 0 | 7.4 |
| 59.7 | 114.9 | 82.7 | 0 | 7.4 |
| 42.3 | 157.2 | 82.4 | 0 | 7.4 |
| 50.6 | 207.7 | 82.0 | 0 | 7.4 |
| 65.2 | 272.9 | 81.4 | 0 | 7.3 |
| 52.9 | 325.8 | 81.0 | 0 | 7.3 |
| 45.5 | 371.3 | 80.5 | 0 | 7.1 |
| 53.4 | 424.7 | 79.6 | 0 | 6.9 |
| 78.0 | 502.6 | 78.7 | 0 | 6.9 |
| 62.3 | 565.0 | 78.4 | 0 | 6.9 |
| 64.0 | 629.0 | 77.7 | 0 | 6.4 |
| 68.1 | 697.1 | 76.1 | 0 | 6.4 |
| 65.6 | 762.7 | 75.4 | 0 | 6.1 |
| 63.9 | 826.6 | 74.7 | 0 | 6.0 |
| 69.5 | 896.2 | 73.7 | 0 | 6.0 |
| 53.7 | 949.9 | 73.1 | 0 | 6.0 |

| | |
|---|---|
| Amount of PA consumed | 11.7 ppm |
| Pounds of PA consumed | 0.012078 lb |
| Pounds of Romaine treated | 949.90 lb |
| Depletion of PA | 0.000013 lb of PA per lb of Romaine |

TABLE 5.2
Reduction of indigenous microorganisms by peroxyacetic acid
with no Lactic acid wash water based on commercial scale test.

| | Aerobic Plate Counts |
|---|---|
| | Log cfu/g |
| Untreated | 3.4 |
| PA Wash Water | 2.7 |
| Log Reduction | 0.7 |

TABLE 5.3
Depletion of test FE sanitizer wash water (Peroxyacetic acid/PA/PAA
with Lactic acid/LA)) in the presence of organic matter based on
commercial scale test.
Product: Diced Romaine Lettuce
Volume of sanitizer 600 gallons
Wash water Temp 38 to 40 F.

| Wt. of Diced | Cumulative Wt. | | | |
|---|---|---|---|---|
| Romaine | of Diced | | | Peroxide |
| added (lb) | Romaine added (lb) | PA (ppm) | LA (ppm) | (ppm) |
| 0.0 | 0.0 | 84.8 | 0 | 7.5 |
| 55.2 | 55.2 | 83.3 | 0 | 7.4 |
| 59.7 | 114.9 | 82.7 | 0 | 7.4 |
| 42.3 | 157.2 | 82.4 | 0 | 7.4 |
| 50.6 | 207.7 | 82.0 | 0 | 7.4 |
| 65.2 | 272.9 | 81.4 | 0 | 7.3 |
| 52.9 | 325.8 | 81.0 | 0 | 7.3 |
| 45.5 | 371.3 | 80.5 | 0 | 7.1 |
| 53.4 | 424.7 | 79.6 | 0 | 6.9 |
| 78.0 | 502.6 | 78.7 | 0 | 6.9 |
| 62.3 | 565.0 | 78.4 | 0 | 6.9 |
| 64.0 | 629.0 | 77.7 | 0 | 6.4 |
| 68.1 | 697.1 | 76.1 | 0 | 6.4 |
| 65.6 | 762.7 | 75.4 | 0 | 6.1 |
| 63.9 | 826.6 | 74.7 | 0 | 6.0 |
| 69.5 | 896.2 | 73.7 | 0 | 6.0 |
| 53.7 | 949.9 | 73.1 | 0 | 6.0 |

| | |
|---|---|
| Amount of PAA consumed | 10.7 ppm |
| Pounds of PAA consumed | 0.011 lb |
| Pounds of Romaine treated | 4011 lb |
| Depletion of PAA | 0.0000028 lb of PAA per lb of Romaine |

TABLE 5.4
Reduction of indigenous microorganisms by FE sanitizer wash water
(Peroxyacetic acid with Lactic acid) based on commercial scale test.

| | Aerobic Plate Counts |
|---|---|
| | Log cfu/g |
| Untreated | 5.1 |
| FE Wash Water | 2.5 |
| Log Reduction | 2.6 |

TABLE 5.5
Depletion of 10 ppm chlorinated wash water in the presence
of organic matter based on commercial scale test.
Product: Diced Romaine Lettuce
Volume of sanitizer 600 gallons
Wash water Temp 38 to 40 F.

| Wt. of Diced | | | |
|---|---|---|---|
| Romaine | Cumulative Wt. of Diced | | |
| added (lb) | Romaine added (lb) | pH | Free Chlorine ppm |
| 0 | 0.0 | 7.1 | 7.6 |
| 286.5 | 286.5 | 7.8 | 1.2 |

| | |
|---|---|
| Amount of free chlorine consumed | 6.4 ppm |
| Pounds of free chlorine consumed | 0.006594 lb |
| Pounds of Romaine treated | 287 lb |
| Depletion of free chlorine | 0.000023 lb of free chlorine |
| | per lb of Romaine |

TABLE 5.6
Reduction of indigenous microorganisms by chlorinated wash
water based on commercial scale test.

| | Aerobic Plate Counts |
|---|---|
| | Log cfu/g |
| Untreated | 5.1 |
| Chlorinated Water | 3.9 |
| Log Reduction | 1.2 |

The depletion of peroxyacetic acid in the FE sanitizer was 5-fold (500%) less than that of the peroxyacetic acid solution with no addition lactic acid. This shows that under the same volume and concentration of peroxyacetic acid, the tested FE sanitizer could disinfect 5 times more produce than the peroxyacetic acid sanitizer with no lactic acid addition. In addition the lbs of free chlorine required to treat a pound of Romaine was 8.5 folds (850%) more than that of the tested FE sanitizer thus indicating that per pound of the tested FE sanitizer could disinfect 8.5 times more produce than per pound of chlorinated water.

The log₁₀ reduction of indigenous microorganism on the Romaine leaf for 73-84 ppm peroxyacetic acid wash water, FE sanitizer wash water (59 to 69 ppm PA and 2,389 to 2,724 ppm LA), and 1.2 to 7.6 ppm free chlorine wash water was 0.7, 2.6, and 1.2-log₁₀, respectively. Although the FE sanitizer in the study was below the optimum lower limit, its log₁₀ reduction on indigenous microorganisms attached on the Romaine leaf was still 2.2 and 3.7 fold, respectively, higher than that of the chlorinated water and peroxyacectic acid wash water.

### Example 6

Results for Chicken Skin

Inoculum preparation: ATCC freeze dried culture was rehydrated in 10 mL of sterilized TSB and mixed homogeneously. 0.1 mL of the stock solution was transferred to 10 mL of TSB and incubated at 37 C for 24 h. Enrichment was streaked to confirm purity. 2 mL of the enriched stock was transferred to 200 mL of TSB and incubated at 37 C for 24 h for E. coli K-12 (ATCC 25253) (EC) resulting in ˜108 cfu/mL stationary phase culture stock. The stock was cooled at 4 C for 1 h. Microbial population of the stationary phase stock culture was enumerated by means of serial dilution with 9-mL Butterfield phosphate buffer tubes and spread plating on Tryptic Soy Agar (TSA) pre-poured agar plates for EC

Chicken wing surface inoculation: The 200 mL ˜10⁸ cfu/mL stock culture solution was homogeneously mixed by shaking and swirling the Erlenmeyer flask. The 200 mL culture was separated into 4 centrifuge tubes (50 mL each) and centrifuged at 10,000 rpm and 4 C for 15 min. The stock culture pellet was re-suspended with 5 mL of 5% Horse Serum. All the re-suspended cultures from the four centrifuge tubes were combined to form 20 mL ˜10⁹ cfu/mL inoculating stock culture. The chicken middle wing was pat dried by paper towel. A permanent marker was used to mark a 2.5 cm×2.5 cm square at the middle of the flat surface of the chicken wing for inoculation. 50 uL inoculum in the form of 2 uL droplets were spiked onto the marked square on the chicken wing skin. The inoculated chicken wing was placed into a sterile polypropylene basket. The spiked chicken wing inside the polypropylene basket was dried inside a biological safety cabinet for 120 min at 18 to 24 C

Treatment of inoculated chicken wing: Each sample of inoculated chicken wing was placed into a 2.5 L stomacher bag containing 500 mL of the tested solution for 10 minutes with intermittent 30 s-shakings at 0, 4, and 9 min. The tested solutions included city water, lactic acid solution (LA), peracetic acid solution (PA), and FreshRinse solution(FR). The treated chicken wing was immediately removed from the treatment solution after 10 min and placed into a sterilized Petri-dish, with the marked side facing up. A pre-wet sterilized swab dipped in 10 mL Butterfield phosphate buffer with sodium thiosulfate was used to swab the marked area. After swabbing the cotton wool swab was immediately placed back into the 10 mL Butterfield phosphate buffer with sodium thiosulfate and mixed. One mL was immediately transferred from the aforementioned tube to a 9 mL Butterfield phosphate buffer. The total treatment time including the exposure time, the swabbing time, and the transfer time was 10.5 min. Each solution treatment was performed in duplications. The reduction for each solution treatment was compared to that of the city water treatment

TABLE 6.1
Log reductions of E. coli K-12 (ATCC 25253) attached on chicken
wing by PA solution (40, 60, and 80 ppm), LA solution (5000 and
10,000 ppm), and FR solution (5000 ppm LA + 40 ppm PA,
5000 ppm LA + 60 ppm PA, 5000 ppm LA + 80 ppm PA,
10,000 ppm LA + 40 ppm PA, 10,000 ppm LA + 60 ppm PA,
and 10,000 ppm LA + 80 ppm PA):

| Middle chicken wing skin | | Peracetic acid (PA) (ppm) | | | |
|---|---|---|---|---|---|
| 10.5 min residence time | | 0 | 40 | 60 | 80 |
| Lactic Acid | 0 | | 0.6 | 0.5 | 1.0 |
| (LA) (ppm)) | 5,000 | 0.3 | 1.5 | 1.5 | 1.8 |
| | 10,000 | 0.7 | 2.7 | 2.7 | 3.7 |

### Example 7

Results for Strawberries and Potatoes

ATCC freeze dried culture was rehydrated in 10 mL of sterilized tryptic soy broth and mixed homogeneously. 0.1 mL of the stock solution was transferred to 10 mL of TSB and incubated at 37 C for 24 h. Enrichment was streaked to confirm purity. 2 mL of the enriched stock was transferred to 200 mL of TSB and incubated at 37 C for 24 h and 36 h, respectively for E. coli K-12 (ATCC 25253) (EC) and Listeria innocua (ATCC 33090) (LI) resulting in ˜10⁸ cfu/mL stationary phase culture stock. The stock was cooled for at 4 C for 1 h. Microbial population of the stationary phase stock culture was enumerated by means of serial dilution with 9-mL Butterfield phosphate buffer tubes and spread plating on Tryptic Soy Agar (TSA) pre-poured agar plates for EC or Modified Oxford Agar (MOX) pre-poured agar plates for LI.

Fruit surface inoculation: The 200 mL ˜10⁸ cfu/mL stock culture solution was homogeneously mixed by shaking and swirling the Erlenmeyer flask. The 200 mL culture was separated into 4 centrifuge tubes (50 mL each) and centrifuged at 10,000 rpm and 4 C for 15 min. The stock culture pellet was re-suspended with 5 mL of 5% Horse Serum. All the re-suspended cultures from the four centrifuge tubes were combined to form 20 mL ˜10⁹ cfu/mL inoculating stock culture. The outer surface of the test item was cut into 5 cm×2.5 cm pieces or diced into 10 cm cubes in the case of tomatoes for inoculation. 50 uL inoculum in the form of 2 uL droplets were spiked onto the strawberry, while 100 uL in the form of 2 uL droplets were inoculated onto the potato peel. The spiked surfaces or peels were placed in a biological safety cabinet for 60 min at 18 to 24 C for drying.

Treatment of inoculated surfaces: Each sample of inoculated surface was placed into a 2.5 L stomacher bag containing 500 mL of the tested solution and shook vigorously for 45 s. The tested solutions included city water, lactic acid solution (LA), peracetic acid solution (PA), and FreshRinse solution (FR). The treated surface was immediately transferred into a sterile stomacher bag containing 100mL of Butterfield phosphate buffer with 1 sodium thiosulfate pellet at the end of the 45 s treatment. Each solution treatment was performed in duplications. The reduction for each solution treatment was compared to that of the city water treatment.

TABLE 7.1
Log reductions of E. coli K-12 (ATCC 25253) attached on potato
peel by PA solution (60 and 80 ppm), LA solution (1500 and
3000 ppm), and FR solution (1500 ppm LA + 60 ppm PA, 1500 ppm
LA + 80 ppm PA, 3000 ppm LA + 60 ppm PA, and 3000 ppm
LA + 80 ppm PA):

| | | Peracetic acid | | |
|---|---|---|---|---|
| Potato peel | | (PA) (ppm) | | |
| 45 sec residence time | | 0 | 60 | 80 |
| Lactic Acid | 0 | | 1.0 | 1.2 |
| (LA) (ppm)) | 1,500 | 0.1 | 2.2 | 2.3 |
| | 3,000 | 0.0 | 2.0 | 2.1 |

TABLE 7.2
Log reductions of Listeria innocua (ATCC 33090) attached on whole
strawberry surface by PA solution (50 ppm), LA solution (1500 and
3000 ppm), and FR solution (1500 ppm LA + 50 ppm PA and
3000 ppm LA + 50 ppm PA):

| | |
|---|---|
| | Peracetic |
| | acid |
| Whole Strawberry | (PA) (ppm) |

| | | |
|---|---|---|
| 45 sec residence time | 0 | 50 |

| | | | |
|---|---|---|---|
| Lactic Acid | 0 | | 1.1 |
| (LA) (ppm)) | 1,500 | 0.0 | 1.9 |
| | 3,000 | 0.5 | 2.6 |

All publications, patents and patent applications cited herein, whether supra or infra, are hereby incorporated by reference in their entirety to the same extent as if each individual publication, patent or patent application was specifically and individually indicated to be incorporated by reference.

1. A method of treating skin or mucosa comprising contacting the surface with an aqueous composition which comprises:
i) an organic peracid of the formula RC(O)OOH wherein R is methyl, ethyl, n-propyl, or s-propyl;
ii) a 2-hydroxy organic acid selected from tartaric acid, citric acid, malic acid, mandelic acid, and lactic acid; and, optionally, a surfactant;
wherein the aqueous composition has a pH from 2.5 to 7.8, inclusive and the concentration of peracid is from 40 to 250 ppm (w/w) inclusive, and the concentration of the 2-hydroxy organic acid is from 0.1 to 1% (w/w), inclusive.

2. The method of claim 1, wherein the composition also comprises an anionic surfactant.

3. The method of claim 1, wherein the peracid is peroxyacetic acid and the 2-hydroxy organic acid is L-(+)-lactic acid.

4. The method of claim 3, wherein the concentration of the peroxyacetic acid in the composition is from 50 to 100 ppm (w/w), the concentration of the lactic acid in the composition is from 0.1% to 0.6% (w/w).

5. The method of claim 3, wherein concentration of peroxyacetic acid in the composition is from 60 to 80 ppm (w/w), the concentration of lactic acid in the composition is from 0.1% to 0.4% (w/w).

6. The method of claim 3, wherein the pH is between 2.5 and 4.5.

7. The method of claim 1, wherein the pH is from 2.8 to 3.2.

8. The method of claim 1, wherein the pH is about 3.0.

9. The method of claim 1, wherein the composition is at a temperature of 35° F. to 45° F.

10. The method of claim 1, wherein the composition is substantially free of nonionic surfactants, cationic surfactants or anionic surfactants

11. The method of claim 1, wherein the composition which is a solution, a paste, a gel, a foam, or a suspension.

12. (canceled)

13. (canceled)

14. The method of claim 1, wherein the contacting is for a period of time is from 10 seconds to 2 minutes.

15-17. (canceled)

18. The method of claim 1, wherein the concentration of peroxyacetic acid is 70 to 80 ppm (w/w), and the concentration of the lactic acid is from 0.2 to 0.4% (w/w).

19. The method of claim 1, wherein the composition is at temperature selected from room temperature, ambient temperature, or from 35° F. to 85° F.

20. (canceled)

21. The method of claim 1, wherein the composition is substantially free of nonionic surfactants, cationic surfactants and anionic surfactants.

22. (canceled)

23. The method of claim 1, wherein the treatment sanitizes the surface by killing or inhibiting the growth of bacteria on, or attached to, the surface of the skin or mucosa.

24. (canceled)

25. The method of claim 1, wherein the composition further comprises an emollient.

26. A human personal care product comprising an aqueous composition comprising:
i) an organic peracid of the formula RC(O)OOH wherein R is methyl, ethyl, n-propyl, or s-propyl;
ii) a 2-hydroxy organic acid selected from tartaric acid, citric acid, malic acid, mandelic acid, and lactic acid; and, optionally, a surfactant,
wherein the aqueous composition has a pH from 2.5 to 7.8, inclusive and the concentration of peracid is from 40 to 250 ppm (w/w) inclusive, and the concentration of the 2-hydroxy organic acid is from 0.1 to 1% (w/w), inclusive; and
iii) and an emollient.

27. The product of claim 26 which is a liquid soap, a hand sanitizes, a cleansing wipe, body wash, acne treatment product, or shampoo.
